(19) United States
(12) Patent Application Publication
(10) Pub. No.: US 2005/0153435 A1
(43) Pub. Date: Jul. 14, 2005

(54) Multiple sample screening using a silicon substrate

(75) Inventor: William B. Archibald, Foster City, CA (US)

Correspondence Address: MARTINE PENILLA & GENCARELLA, LLP, 710 LAKEWAY DRIVE, SUITE 200, SUNNYVALE, CA, 94085, US

(73) Assignee: Solus Biosystems, Inc., Palo Alto, CA (US)

(21) Appl. No.: 11/039,276

(22) Filed: Jan. 18, 2005

Related U.S. Application Data
(63) Continuation-in-part of application No. PCT/US03/37387, filed on Nov. 21, 2003.
(63) Continuation-in-part of application No. 10/366,464, filed on Feb. 14, 2003.
(60) Provisional application No. 60/428,241, filed on Nov. 22, 2002.
(60) Provisional application No. 60/356,111, filed on Feb. 14, 2002.

(51) Int. Cl. C12Q 1/68; C12M 1/34

(52) U.S. Cl. 435/287.2

(57) ABSTRACT

A silicon substrate for enabling the analysis of a biological sample is provided which includes the silicon substrate having an active surface and a backside surface where the active surface of the silicon substrate has a plurality of recessed regions defined therein. The recessed region has a probe region on one side of the recessed region where each one of the plurality of recessed regions is defined as an elongated well which is substantially rectangular. The plurality of recessed regions is configured for receiving a plurality of biological samples with a complimentary probe region on an opposite side of each one of the plurality of recessed regions and a sample receiving region being between the probe region and the complimentary probe region. The sample receiving region is capable of receiving the biological sample for analysis and the complimentary probe region is capable of interfacing with an electrically conductive probe for enabling the analysis. The silicon substrate is disposed on a substrate holder and the silicon substrate is between about 1 micron and 4 cm thick and each one of the plurality of recessed regions is defined as a rectangular well having a length of about 5 mm, a width of about 125 microns, and a depth of about 25 microns. The plurality of recessed regions includes 10 capillaries.

### CROSS REFERENCE TO RELATED APPLICATION

This application is a continuation-in-part and claims 35 U.S.C. §§ 120 and 365(c) priority from co-pending International Patent Application No. PCT/US2003/037387 filed on Nov. 21, 2003 which designates the United States of America and is entitled “High Throughput Screening with Parallel Vibrational Spectroscopy,” which claims priority from a U.S. Provisional Patent Application No. 60/428,241 filed on Nov. 22, 2002, both of which are incorporated herein by reference in their entirety.

This patent application is also a continuation-in-part and claims 35 U.S.C. § 120 priority from co-pending U.S. patent application Ser. No. 10/366,464 entitled “High Throughput Screening with Parallel Vibrational Spectroscopy” filed on Feb. 14, 2003 which claims priority from U.S. Provisional Application No. 60/356,111 filed on Feb. 14, 2002, both of which are incorporated herein by reference in their entirety.

This application is related to U.S. patent application Ser. No. _______ (Attorney Docket No. SBIOP001A) entitled “Multiple Sample Screening using IR Spectroscopy” filed on ______. This application also related to U.S. patent application Ser. No. ______ (Attorney Docket No. SBIOP001B) entitled “Method for Multiple Sample Screening using IR Spectroscopy” filed on ______. The aforementioned patent applications are herein incorporated by reference in their entirety.

### BACKGROUND OF THE INVENTION

1. Field of the Invention

The present invention relates to screening of fluid samples using optical analysis and, more particularly, to simultaneous multiple sample screening using vibrational spectroscopy.

2. Description of the Related Art

Virtually every area of the biomedical sciences needs to determine the presence, structure, and function of particular analytes that participate in chemical and biological interactions. The needs range from the basic scientific research lab, where biochemical pathways are being mapped and correlated to disease processes, to clinical diagnostics, where patients are routinely monitored for levels of clinically relevant analytes. Other areas include pharmaceutical research, military applications, veterinary, food, and environmental applications. In all of these cases, the presence, quantity, and structure activity relationships of a specific analyte or group of analytes needs to be determined.

Numerous methodologies have been developed to meet this need. The methods include enzyme-linked immunosorbent assays (ELISA), radio-immunoassays (RIA), numerous fluorescence assays, mass spectrometry, colorimetric assays, gel electrophoresis, as well as a host of more specialized assays. Most of the assay techniques require specialized preparations such as chemically attaching a label or purifying and amplifying a sample to be tested. Generally, an interaction between two or more molecules is monitored via a detectable signal relating to the interaction. Typically a label conjugated to either a ligand or anti-ligand of interest generates the signal. Physical or chemical effects produce detectable signals. The signals may include radioactivity, fluorescence, chemiluminescence, phosphorescence, and enzymatic activity. Spectrophotometric, radiometric, or optical tracking methods can be used to detect many labels.

Unfortunately, in many cases it is difficult or even impossible to label one or all of the molecules needed for a particular assay. The presence of a label may interrupt molecular interaction or otherwise make the molecular recognition between two molecules not function for many reasons including steric effects. In addition, none of these labeling approaches can determine the exact nature of the interaction. Active site binding to a receptor, for example, is indistinguishable from non-active site binding, and thus no functional information is obtained from the present detection methodologies. A method to detect interactions that eliminates the need for the label and that yields functional information would greatly improve upon the above mentioned approaches.

The term “molecular interaction” means any interaction, including binding and biochemical interactions between at least two molecules. Binding interactions include for example binding between antibody binding site and antigen, binding between a protein and a ligand, such as between a membrane protein and an effector that binds the protein, and interactions determined indirectly by intracellular changes that occur upon addition of chemical substances that may act by binding to a cell membrane receptor, binding to effectors that bind to cell membrane receptors, thereby preventing effector binding to their receptors, and intracellular entry of a molecule that leads to some detectable change in another molecule or cellular process.

Detection technology is commercially very important. The biomedical industry relies on tests for a variety of water-based or fluid-based physiological systems to evaluate protein-protein interactions, drug-protein interactions, small molecule binding, enzymatic reactions, and to evaluate other compounds of interest. Unfortunately, typical assay techniques require highly specific probes, such as specific antibodies.

Vibrational spectroscopy is a well established, non-destructive, analytical tool that can reveal much information about molecular interactions. Infrared spectroscopy involves the absorption of electromagnetic radiation generally between 0.770-1000 microns, which represent energies on the order of those found in the vibrational transitions of molecular species. Variations in the positions, widths, and strengths of these modes with composition and structure allow identification of molecular species. One advantage of infrared spectroscopy is that virtually any sample, in virtually any state, can be studied without the use of a separate label. Liquids, solutions, pastes, powders, films, fibers, gases, and surfaces can be examined by a judicious choice of sampling techniques.

Unfortunately, these systems suffer sensitivity and/or speed limitations. The number of photons that can interact with the sample in a short time to generate a meaningful signal decreases dramatically as sample sizes increase and generally limits both sensitivity and speed. A solution to this problem would open up new areas of discovery and would be particularly important in the burgeoning field of combinatorial chemistry, which would benefit greatly by usage of a rapid assay of huge numbers of very tiny samples.

### SUMMARY OF THE INVENTION

Broadly speaking, the present invention is a method and apparatus that enables analysis of multiple samples using vibrational spectroscopy. It should be appreciated that the present invention can be implemented in numerous ways, including as a process, an apparatus, a system, a device or a method. Several inventive embodiments of the present invention are described below.

In one embodiment, a silicon substrate for enabling the analysis of a biological sample is provided which includes the silicon substrate having an active surface and a backside surface where the active surface of the silicon substrate has a plurality of recessed regions defined therein. The recessed region has a probe region on one side of the recessed region where each one of the plurality of recessed regions is defined as an elongated well which is substantially rectangular. The plurality of recessed regions is configured for receiving a plurality of biological samples with a complimentary probe region on an opposite side of each one of the plurality of recessed regions and a sample receiving region between the probe region and the complimentary probe region. The sample receiving region is capable of receiving the biological sample for analysis and the complimentary probe region is capable of interfacing with an electrically conductive probe for enabling the analysis. The silicon substrate is disposed on a substrate holder and is between about 1 micron and 4 cm thick and each one of the plurality of recessed regions is defined as a rectangular well having a length of about 5 mm, a width of about 125 microns, and a depth of about 25 microns. The plurality of recessed regions includes 10 capillaries.

The advantages of the present invention are numerous, most notably the embodiments enable screening of multiple samples using isoelectric focusing and IR spectroscopy. Specifically, samples in capillaries in a wafer can be separated according to their electrical charges by using isoelectric focusing. The isoelectric focusing moves the samples along the capillaries to certain locations to form bands. Once the samples have settled in a location in a portion of the capillaries, IR light from an interferometer is transmitted through the capillaries. A camera can detect and record the IR light absorption by the bands in each of the capillaries. The data from the camera can be processed by using Fourier transform to generate an IR absorption spectrum for each of the bands. By using the IR absorption spectrum, the samples in the capillaries may be characterized.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

The present invention will be readily understood by the following detailed description in conjunction with the accompanying drawings. To facilitate this description, like reference numerals designate like structural elements.

FIG. 1 shows an example of a reflectance mode apparatus in accordance with one embodiment of the present invention.

FIG. 2 shows an example of a transmission mode apparatus in accordance with one embodiment of the present invention.

FIG. 3A shows sample holder having three sampling units constructed with infrared transparent material.

FIG. 3B shows a sample holder that includes non-transparent matrix regions in accordance with one embodiment of the present invention.

FIG. 4A depicts a multiple sample analyzing system in accordance with one embodiment of the present invention.

FIG. 4B shows a more detailed block diagram of the multiple sample analyzing system in accordance with one embodiment of the present invention.

FIG. 5A shows a detailed diagram of the multiple sample analyzing system in accordance with one embodiment of the present invention.

FIG. 5B illustrates an interferometer in accordance with one embodiment of the present invention.

FIG. 6 shows a read head and a write head in accordance with one embodiment of the present invention.

FIG. 7A shows a read head in accordance with one embodiment of the present invention.

FIG. 7B depicts a side view of the read head in accordance with one embodiment of the present invention.

FIG. 7C illustrates a side view of the wafer attached to a wafer holder in accordance with one embodiment of the present invention.

FIG. 7D illustrates a top view of the wafer holder in accordance with one embodiment of the present invention.

FIG. 8A shows a cross-sectional view of the read head attached to a sample holder in accordance with one embodiment of the present invention.

FIG. 8B illustrates a close-up view of the read head connecting with the wafer holder in accordance with one embodiment of the present invention.

FIG. 9A shows a top view of the wafer that is configured to include recesses where samples can be inputted and analyzed in accordance with one embodiment of the present invention.

FIG. 9B illustrates a top of view of an alternative wafer in accordance with one embodiment of the present invention.

FIG. 9C shows a top view of a wafer with extended length capillaries in accordance with one embodiment of the present invention.

FIG. 10A shows a side view of the capillary in accordance with one embodiment of the present invention.

FIG. 10B illustrates the write head inputting a fluid sample into the capillary in accordance with one embodiment of the present invention.

FIG. 10C depicts an oval shaped recessed region in accordance with one embodiment of the present invention.

FIG. 10D illustrates a square shaped recessed region in accordance with one embodiment of the present invention.

FIG. 10E shows a round shaped recessed region in accordance with one embodiment of the present invention.

FIG. 11 illustrates an imaging process that reveals molecular details such as location, movement, and binding of solutes from sample introduced to an isoelectric separation chamber in accordance with one embodiment of the present invention.

FIG. 12A depicts a sample that has been analyzed through IR spectroscopy in accordance with one embodiment of the present invention.

FIG. 12B shows a close-up view of the IR light absorption spectrum for a particular sample in accordance with one embodiment of the present invention.

FIG. 13 illustrates a top view of the capillary in accordance with one embodiment of the present invention.

FIG. 14 shows a side view of the wafer in accordance with one embodiment of the present invention.

FIG. 15 depicts a source plate in accordance with one embodiment of the present invention.

FIG. 16A shows a detection field of a camera in accordance with one embodiment of the present invention.

FIG. 16B illustrates an exemplary pixel pattern of a portion of the detection field of the camera in accordance with one embodiment of the present invention.

FIG. 17A illustrates a Fourier transform of data shown on a graph where camera output is plotted against time in accordance with one embodiment of the present invention.

FIG. 17B depicts graphs that show an absorption spectrum of one band in a first capillary and a second capillary in accordance with one embodiment of the present invention.

FIG. 17C illustrates a graph that shows the IR absorption spectrum of only the sample as discussed in FIG. 17B in accordance with one embodiment of the present invention.

FIG. 18 shows a flowchart defining a method for examining a fluid sample in accordance with one embodiment of the present invention.

FIG. 19 illustrates a flowchart which defines a method where samples are analyzed using the multiple sample analyzing system in accordance with one embodiment of the present invention.

FIG. 20 depicts a flowchart which defines a detailed process whereby a biological sample is examined and identified in accordance with one embodiment of the present invention.

### DETAILED DESCRIPTION

An invention, a method and apparatus that enables analysis of multiple biological samples using vibrational spectroscopy, is disclosed. In the following description, numerous specific details are set forth in order to provide a thorough understanding of the present invention. It will be understood, however, by one of ordinary skill in the art, that the present invention may be practiced without some or all of these specific details. In other instances, well known process operations have not been described in detail in order not to unnecessarily obscure the present invention.

In general terms, the present invention includes methods and apparatuses for using charge based separation and IR spectroscopy on biological samples in each of a plurality of capillaries in a wafer. In one embodiment, a sample is inputted into a capillary of the wafer, and components within the sample are separated using isoelectric focusing. IR light from an interferometer is then applied to the wafer. The IR light that has moved through the samples in the capillaries is received and captured by an infrared camera. The infrared camera then transmits the captured IR image to a processor which can apply inverse Fourier transform to the data to derive an IR absorption spectrum of each of the components in the sample. This may be done concurrently with all of the samples in the capillaries on the wafer. Consequently, concurrent testing of multiple samples may be conducted in a consistent testing environment thereby increasing testing efficiency and accuracy.

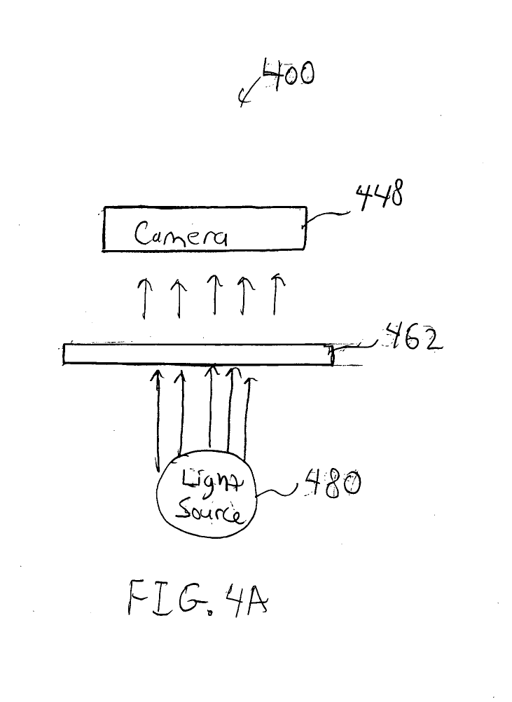

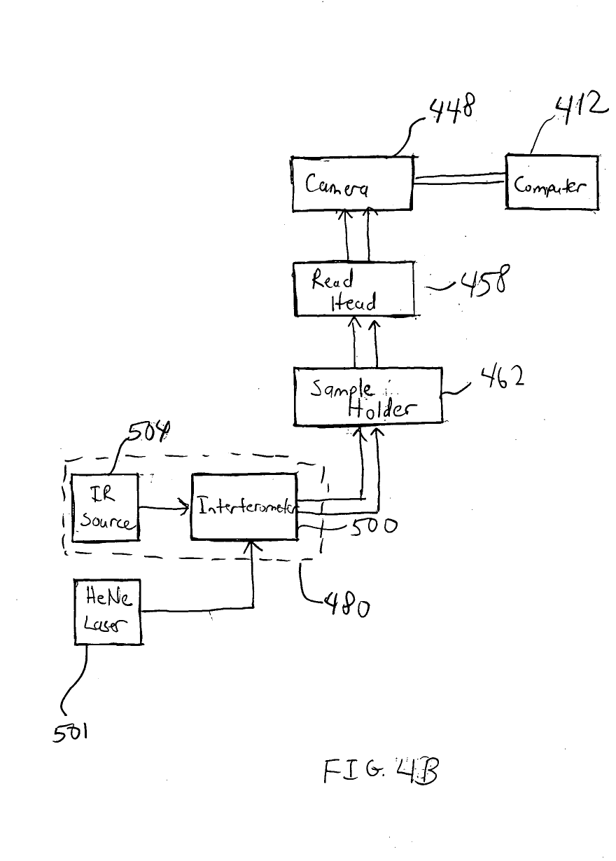

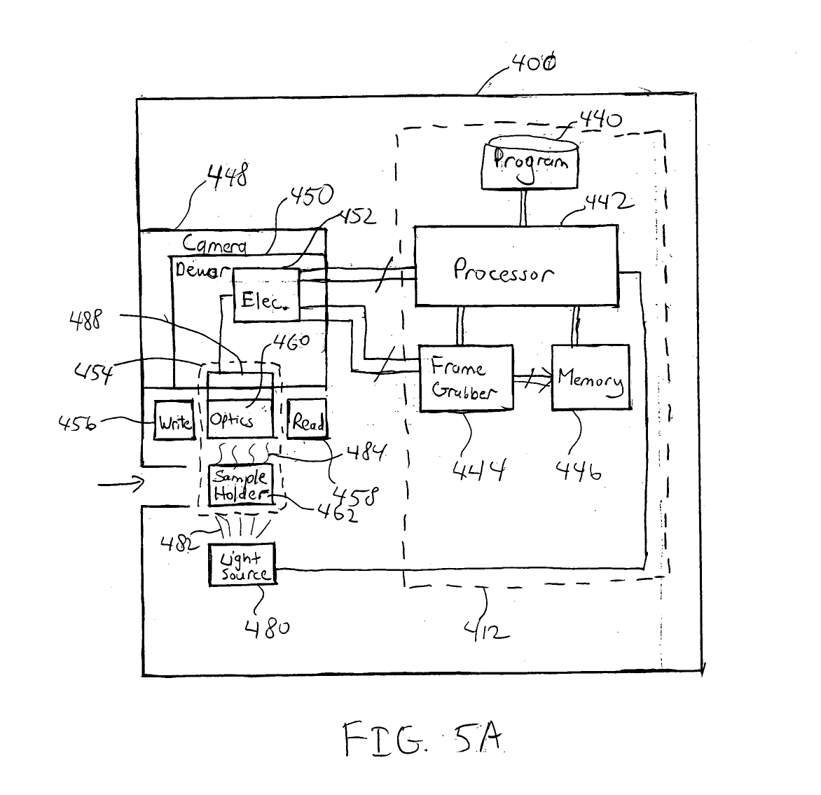

The following discussion up to FIG. 4A disclose various ways of examining multiple samples in a substantially concurrent manner. FIGS. 4A through 20 concentrate the discussion on methods and apparatuses for using both isoelectric focusing and IR light transmission/absorption to concurrently characterize biological components within multiple samples.

The inventor studied the problem of multiple sample spectroscopy with a total system viewpoint and realized that the quantity of light processed per sample is a major limitation to the assay of many small samples simultaneously. That is, the spectroscopic analysis of a large number of samples in parallel requires a much higher flow of total light to obtain parallel information for each sample simultaneously. This system obstacle may be addressed by one or more of: i) increasing the amount of starting light with parabolic optics and multiple light sources; ii) adopting a high bandwidth system that uses wide spectrum light and Fourier analysis, allowing much higher light fluxes and consequent information flow; iii) discovery of capillary and alternative sample formats that greatly increase light throughput while permitting large sample numbers; iv) discovery of miniature sample holder designs that can be mass produced by semiconductor processing techniques; and v) discovery of biochemical and cellular focusing techniques that further optimize signal energy use for improved signal to noise. Each of these discoveries contributes to improved performance, singly and in combination, and facilitates the use of higher sample number spectroscopic assays, as further detailed below.

Embodiments of the invention utilize light spectra of multiple wavelengths to measure absorption and/or transmission spectra from arrays of multiple samples simultaneously. In contrast to many previous techniques, the high bandwidth systems of embodiments of the present invention use entire spectral regions, combined with Fourier analysis, for much greater total light usage and real time detection of individual wavelengths without requiring narrow light filtering. Most other spectroscopic systems discard the vast majority of light from a light source via bandpass filtering or by use of a diffraction grating and selection of a wavelength. The high bandwidth and Fourier analysis are particularly desirable in combination with prismatic structures and small sized but high sample number assay targets.

The term “prismatic” means to bend light used in an optical measurement with respect to the surface of a target transparent medium such that the light enters the surface at an angle closer to the perpendicular of the target surface. A light transparent prism may be used in a prismatic fashion by choosing suitable angles and placement of the prism near to or in contact with the target.

Fourier transform methods used in embodiments of the invention are known and have been used for spectroscopy and for total internal reflectance as exemplified in U.S. Pat. No. 5,416,325 issued to Buontempo et al., May 16, 1995. The contents of this patent, and particularly the described methods for maximizing the ratio of signal to noise for low light intensity signals specifically are incorporated by reference in their entireties. The contents of U.S. Pat. No. 5,777,736 issued to Horton on Jul. 7, 1998; U.S. Pat. No. 5,254,858 issued to Wolfman et al. on Oct. 19, 1993; U.S. Pat. No. 4,382,656 issued to Gilby on May 10, 1983; U.S. Pat. No. 4,240,692 issued to Winston on Dec. 23, 1980; U.S. Pat. No. 4,130,107 issued to Rabl et al. on Dec. 19, 1978; and U.S. Pat. No. 5,361,160 issued to Normandin et al. on Nov. 1, 1994 also provide details for use of Fourier transform spectroscopic methods are particularly incorporated by reference, and represent art known to the skilled artisan.

Light from a light source is modulated and an interferometer for this purpose preferably is used within a light passageway having focusing and/or beam steering optics to manage the light beam. The managed beam contacts (by reflection or transmission) each sample simultaneously and then is directed toward the detector, which preferably is a two dimensional detector. The detector collects data simultaneously from the samples and transfers the data to a computer for storage and processing.

The interferometer may be placed on the source side to interrupt the probing light before contact with sample or it may be on the detector side to interrupt the light between the sample and the detector. In either embodiment the interferometer modulates the light prior to detection by the detector. For embodiments that utilize infrared light, as much of the beam path as possible should be in a controlled environment to limit error due to atmospheric absorption. It is highly desirable to control the amount of water vapor and carbon dioxide in the environment surrounding the sample to achieve a stable baseline. Drift in the temperature, humidity, or chemical content of the medium through which the light beam passes during a measurement may change the spectra in an uncontrolled manner. Such change complicates the mathematical subtraction of the background, making it difficult and/or unreliable. In a preferable embodiment dry nitrogen gas is added to spaces where the infrared beam passes on the way to and from a sample.

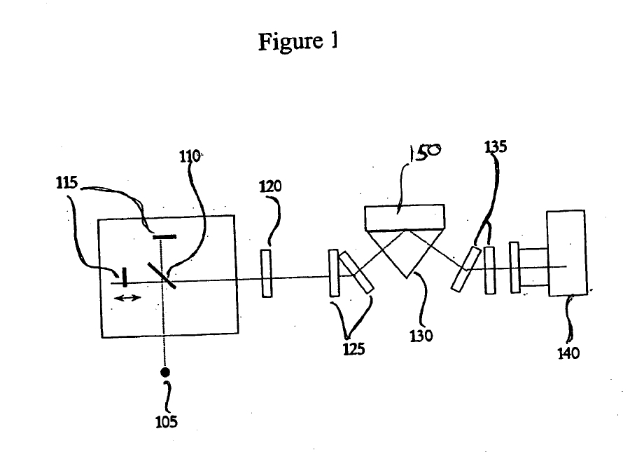

FIG. 1 shows an example of a reflectance mode apparatus in accordance with one embodiment of the present invention. FIG. 1 shows a light source, detector and some parts between the source and detector. Light from light source 105 passes through beam splitter 110 and is reflected by interferometer mirrors 115 into spectral filter 120. Light from spectral filter 120 is focused via focusing and beam steering optics 125 and 130 into the bottom of sample holder 150. The light then interacts with each sample in one or more passes and is then reflected out of sample holder 150 and is focused by optics 135 into infrared camera 140. An embodiment of this system as shown in FIG. 1 comprises six components: 1) source of infrared radiation, 2) a device to modulate the radiation, 3) a sample holder, 4) an infrared detector, 5) steering optics, and 6) a computer to collect, process, and present the spectral data.

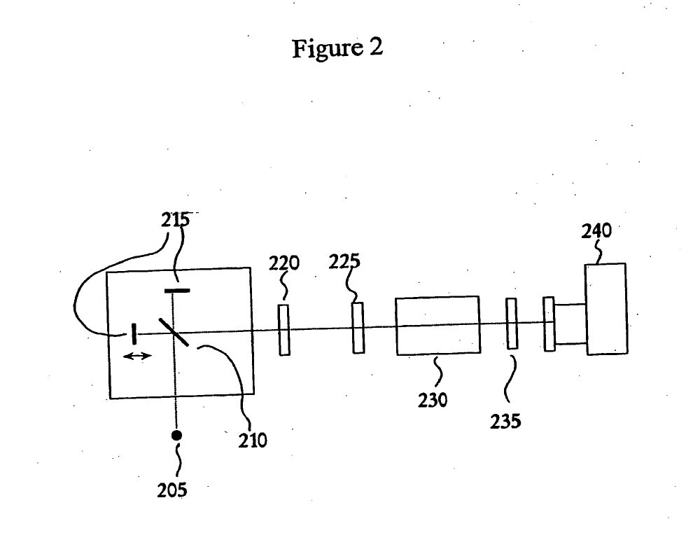

FIG. 2 shows an example of a transmission mode apparatus in accordance with one embodiment of the present invention. Here, radiation from source 205 passes through beam splitter 210 and is reflected by interferometer mirrors 215 into spectral filter 220. Light from spectral filter 220 is focused via focusing optics 225 into the bottom of sample holder 230, where each element of a sample array within holder 230 is illuminated simultaneously. Radiation passes through the samples and then is focused by optics 235 and enters infrared camera 240.

Transmission measurements are carried out by passing light from a source through a sample and to a detector and generally require different sample holders than that used for reflectance measurements. Solution based infrared transmission measurements generally require a short path length transmission cell or a flow-through cell. In both configurations the optical path length through the sample is restricted to short distances such as about 10-50 microns in length for aqueous solutions. A sample may be sandwiched between two infrared transparent windows separated by a thin gasket (Teflon) designed to confine the sample and fix the path length through the sample. A similar sample holder exists where the sample flows through a pipe with an infrared transparent sidewall to let light in and out. Neither configuration allows simultaneous acquisition of infrared absorption spectra from multiple samples. The problems of multiple transmission measurements in parallel can thus be stated as requiring: i) a separation of all samples in an infrared beam; ii) control of the required short path lengths; and iii) reduction of solvent evaporation. These problems were successfully addressed by the discovery of a parallel sample holder design.

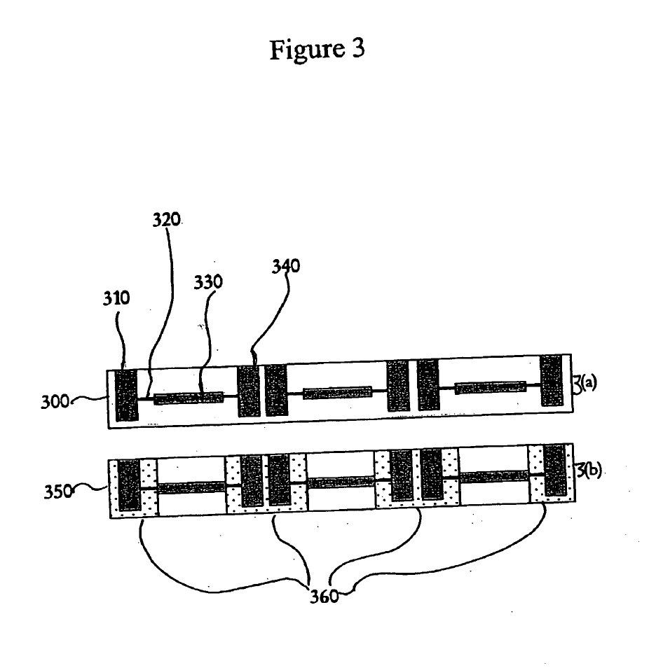

FIG. 3 illustrates a parallel sample holder design in accordance with one embodiment of the present invention. This sample holder has several features that alleviate these problems. First, the holder contains infrared transparent regions to let the beam pass through the sample. These infrared transparent sampling regions may be created by constructing the entire holder from an infrared transparent medium, or by integrating a series of infrared transparent windows into a non-transmitting matrix. Second, the sample holders contain specific sample injection ports, as seen in FIG. 3. Each sample location may have several sample injection ports to allow combination of reactants, solvents, etc. Finally, the sample injection ports are connected to the infrared sampling region by micro channels, which allow the sample to move from the port to the sampling region by capillary action. The capillary fed, short path-length sampling regions can be modified as suited to limit the beam path through the sample and isolation as needed to reduce solvent evaporation.

FIG. 3A shows a side view of a sample holder 300 having three sampling units constructed with infrared transparent material. As seen for the left hand most unit, sample port 310 is used to add or remove a sample or a sample stream that flows through capillary micro channel 320 into sampling region 330 and then out sample port 340.

FIG. 3B shows a sample holder 350 that includes non-transparent matrix regions 360 in accordance with one embodiment of the present invention.

The infrared transparent regions of these sample holders and the sample holders as described below in reference to FIGS. 4A to 20 can be made of one or more infrared transparent materials such as an alkali halide salt (KBr or NaCl), CaF₂, BaF₂, ZnSe, Ge, Si, silicon based materials (e.g., silicon dioxide, etc.), polysilicon, semiconductor materials, crystalline silicon, glass, sapphire, quartz, thin polyethylene, polytetrafluoroethylene (PTFE), or specialized infrared materials such as AMTIR and KRS-5. The use of materials such as Si and Ge allow the entire sample array to be microfabricated using lithography and standard semiconductor processing techniques. The non-transmitting matrix can be made of a low cost material such as a plastic, glass, wax, polymers, elastomers, and so on. In one embodiment, a semiconductor substrate as utilized herein is a substrate made out of a material with a non-zero energy gap that separates the conduction band from the valence band. Such exemplary materials may include, for example, Si, Ge, GaAs, ZnSe, and ZnS.

A majority of contemplated applications utilize the accumulating of spectral information in the wavelength range between 5-16.5 microns. Infrared sources emit radiation over a large wavelength range from the visible to the far infrared and embodiments of the invention use the various wavelengths. Infrared wavelengths outside a desired spectral window may adversely affect the measurement through sample heating. Uncontrolled heating in turn causes background (baseline signal) drift and decreases signal to noise ratio of measurements. Therefore, a spectral filter preferably is included to limit the infrared radiation from a source to a bandwidth of interest, and blocks other radiation generated from the source but which is not necessary for a measurement.

Such blocking is particularly valuable when light intensity is increased for small area samples (i.e. high power density applications). An infrared filter can be fabricated by deposition of a thin film(s) of specialized material(s) (metals and semiconductors) onto a infrared transparent substrate. A general discussion can be found in many optical texts, at http://www.ocli.com/pdf-files/products/geninfoinfraredfilters.pdf or in O. S. Heavens Optical Properties of Thin Solid Films 1991, Dover Press, New York.

Modulation, combined with Fourier transform analysis is particularly powerful for improving signal and analysis time. Light from the source preferably is modulated with an interferometer. A preferable interferometer is a Michelson interferometer. Numerous other interferometer designs exist and are suitable. In principle any interferometer that creates an optical path difference will work in one or more embodiments.

Many laboratory based mid-infrared imaging spectrometers utilize a Michelson interferometer to modulate infrared radiation before the radiation interacts with a sample. The Michelson interferometer often is used in commercial FT-IR spectrometers as the “light source” in their systems. The Michelson interferometer uses a moving mirror system to generate an optical path difference between two components of a split light source. The spectral resolution of a two-beam interferometer is based on the overall optical path difference in the interferometer and number of optical path differences at which the detector is read (number of mirror positions measured). The data from each of the optical path differences is converted to an absorption spectrum with the aide of a mathematical (e.g. Fourier) transform algorithm and a computer.

Two beam systems are capable of very wide bandwidths (25,000-13 cm⁻¹) and very high-resolution (.about0.0.005 cm⁻¹) operation, and are particularly described as they are useful in embodiments of the invention. The need to move one or both mirrors complicates time sensitive analysis when the kinetics of the event being measured is on the same time scale as the mirror speed. In other words, the data are averaged over the time needed to sweep one length of the mirror path; speed and resolution are inversely related. Certain two-beam interferometers utilize a step-scan configuration, where the interferometer steps to a fixed optical path difference and scans a small amount (small mirror movement) around that path length.

The influence on imaging systems is even more profound due to the increased time needed to get the data from the array. The array speed generally scales with the size, the smaller arrays being faster, and single pixel detectors (found in FT-IR spectrometers) generally operate at MHz frequencies. A typical 64×64 pixel Hg—Cd—Te array has a maximum frame rate of 3000 Hz. Since an image must be taken for each optical path difference (mirror position), and the spectral resolution is dependent on the number of different mirror positions measured, higher resolution translates into longer times in the imaging sense as well.

Complicating the speed issue further, many chemical and biological reactions require numerous spectra that must be averaged for noise reduction prior to data processing. A typical protein experiment, for example, may require the combination of 100 or more spectra data for mathematical processing via one or more algorithms such as smoothing, derivatizing, curve-fitting, etc.). Embodiments of the invention provide rapid multiple spectra from each sample in an array which increases system performance and provides good sample throughput speeds

One of the largest contributors to noise when taking infrared measurements in aqueous solutions is drift in the background (baseline). This problem may be addressed by generating a background (baseline) measurement and then using that measurement to reference subsequent spectra. In many cases the stored baseline spectrum is subtracted from subsequent spectra. Typically the baseline will change due to changes in temperature or changes in the atmospheric conditions, such as changes to humidity, carbon dioxide content, etc. These changes manifest themselves as an incomplete subtraction or overcompensation of background effects. The drift problem is acute for measurements of dilute concentrations of molecules, where the baseline noise may overcome the desired signal from molecules in solution.

An infrared spectrometer that may be used herein can have a detector sensitive to mid-infrared radiation in the 5 to 17 micron wavelength range. These detectors include such materials as Hg—Cd—Te, DTGS, thermopiles, quantum well infrared photodetectors (QWIP's), as well as many types of cooled and uncooled bolometers. In an imaging or parallel spectrometer, these detectors are found in either linear (1×128, 1×256, etc.) or rectangular arrays (64×64, 128×128, 4×256, etc.). The detector and read-out electronics form the components of an infrared camera. The camera converts the incoming radiation into a spectral image using mathematical transform algorithms on a standard personal computer.

A majority of chemical and biological reactions take place in aqueous or organic solvents that absorb mid-infrared radiation well. For example, strong absorption in the mid-infrared spectral region generally limits the optical path-length to 5-10 microns in aqueous solutions. Conventional one-at-a-time spectrometers typically use three approaches to obtain spectra in these environments. They include, short path length or flow-through cells, total internal reflectance, and solvent evaporation. Each approach is constrained by the need for infrared transparent sample holder(s), or at least regions in the holder that are transparent. Many embodiments described herein address this problem by (in comparison with earlier art) shrinking the sample size and assaying large numbers of samples simultaneously.

Embodiments of the invention provide diagnostic signals obtained by interaction of light with chemical bonding electrons found in molecules of interest. The diagnostic signals form from electric impulses that correspond to detected light signals. A good signal to noise (random electrical background signals) ratio thus is important to obtain rapid measurements because as the measurement time decreases the amount of light processed (and the electrical signal obtained from the light) becomes smaller. Infrared light is used in many embodiments wherein desired spectral processes involve fundamental vibrational resonances of molecules in the mid-infrared region of the light spectrum, which generally is defined as 4000-400 cm⁻¹ (2.5-25 microns). A majority of biological compounds are limited to 1800-600 cm⁻¹ (5.5-16.7 microns).

To generate probing light in the infrared region, a blackbody emission source typically is used such as a “glowbar” (a hot material such as SiC), a sample or scene's intrinsic heat emission, or from solar infrared radiation. Preferred sources include a single glowbar (silicon carbide rod), Nernst glower (cylinder of rare-earth oxides) or an incandescent wire. A source typically may have power outputs of about 50-100 W and a beam diameter of about 4 cm, or a beam power density of about 4 W/cm². This power density can be increased with focusing optics for smaller samples, and reduced when an aperture is placed between the source and the sample. This power density is acceptable for traditional infrared experiments that involve a single sample in the beam path, or small area samples where the beam can be focused to a specific spot. In larger area sampling environments that exist when hundreds of small samples are to be measured simultaneously, broadening the beam to increase the effective area decreases the power density at each location in the sample. Therefore in order maintain an advantageous power density for an increased area of larger samples the infrared source power desirably is increased.

In an embodiment, a spinning mirror interferometer, such as that used for infrared measurements is modified for an increased mirror rotational speed as necessary for the shorter wavelength light. Advances in light modulation technology in the future will provide more convenient alternative methods for generating suitable modulation and are contemplated for embodiments of the invention.

Fluorescence, phosphorescence, time resolved fluorescence and/or chemiluminescence may be used in conjunction with infrared techniques as described here. Drug discovery methods advantageously may utilize such added information to reveal further molecular and metabolic information. The additional information is helpful particularly for biochemical and cellular studies where the effects of a test compound in a sample are very complex and multiple chemical interactions need to be examined. For example, a cell may be genetically engineered to express luciferin and luciferase and generate light from a biochemical pathway and used as a probe in multiple sample wells to test for new lead drug compounds. Effects from the test compounds may be detected as visible light signals. By monitoring both infrared reflectance and visible light signals simultaneously, chemical binding of test compounds to a cell surface can be monitored, and the timing and effect on the biochemical process monitored.

FIGS. 4A through 20 show various embodiments of methods and apparatuses for analyzing multiple chemical/biological samples using vibrational spectroscopy such as, for example, IR spectroscopy. It should be appreciated that the methods and apparatuses can analyze and examine any suitable type of biological samples such as, for example, any suitable type and/or numbers of molecules that are utilized in the biological and chemical sciences. Moreover, it should be appreciated that each biological sample may include any suitable number (e.g., multiple) of sample components (e.g., one or more of a drug, antibody, water, proteins, biological molecules, etc.). In addition, the samples to be analyzed may be in any suitable type of physical state such as, for example, liquid, semi-liquid, semi-solid, solid, powder, etc. In one embodiment, multiple recesses such as, for example, capillaries on an active surface of a silicon chip/wafer are each filled with samples to be analyzed. Then isoelectric focusing is utilized to separate different chemical/biological components contained within the samples. Therefore, an electrical field is applied to the capillary and a pH gradient is generated along the length of the capillaries. Consequently, different molecules within the sample move to different positions along the capillaries where their net charge is zero. The IR light that has passed through the samples is detected by an IR camera which transmits the data to a computer which can perform a Fourier transform on the data thereby generating an IR absorption spectrum. Because certain biological/chemical components (e.g., proteins, genetic materials, protein interaction resultant, etc.) generate a certain IR absorption at different wavelengths, the IR absorption spectrum can be examined to determine what components are in the sample.

FIG. 4A depicts a multiple sample analyzing system 400 in accordance with one embodiment of the present invention. It should be appreciated that the system 400 in FIG. 4A has been simplified for ease of understanding. In one embodiment, the multiple sample analyzing system 400 includes a light source 480 that transmits IR light through a sample holder 462 that contains one more samples to be analyzed. It should be appreciated that the sample may be any suitable sample (e.g., biological, chemical, etc.) that can be analyzed by IR spectroscopy. The IR light that has been transmitted through the sample(s) can be detected by an IR camera 448. By analyzing the optical signals received by the camera 448, IR absorption map such as, for example, an IR absorption spectrum may be generated to determine/characterize the composition of the sample(s) in the sample holder 462. An IR absorption map may be any suitable type of graphical and/or mathematical representation that may show IR light absorption of the sample(s). In one embodiment, the IR absorption data is capable of being displayed as at least one data point based on the detection of the infrared light transmitted through the sample(s). Exemplary embodiments of IR absorption maps are shown below in reference to FIGS. 11C, 12A and 12B.

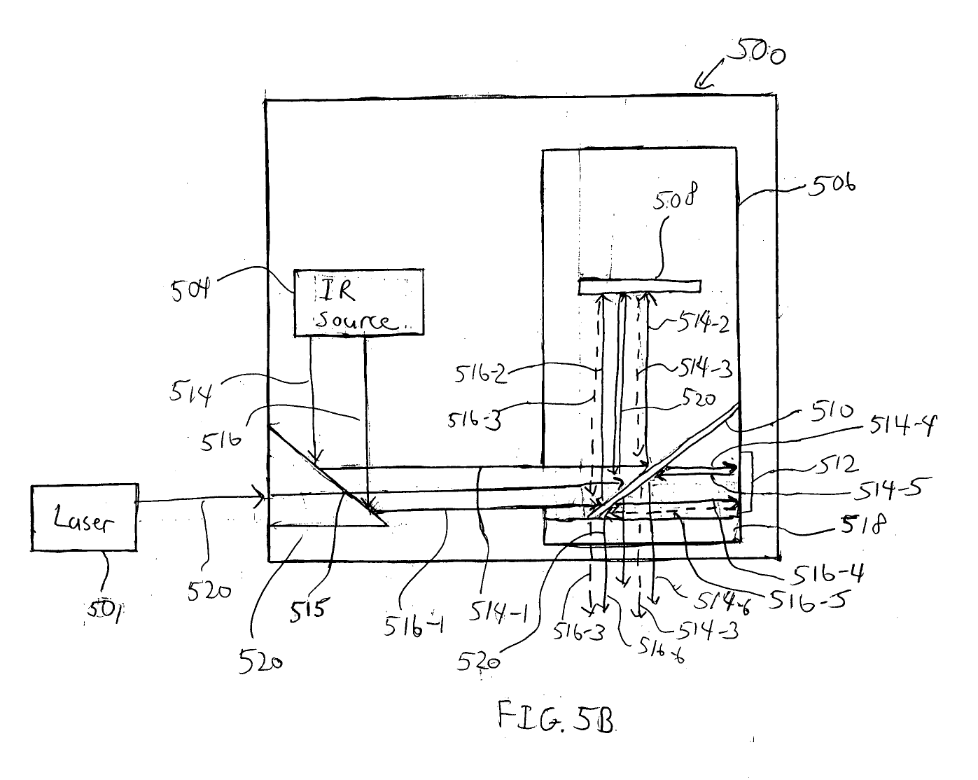

FIG. 4B shows a more detailed block diagram of the multiple sample analyzing system 400 in accordance with one embodiment of the present invention. In one embodiment, the multiple sample analyzing system 400 includes the IR source 504 that transmits light into an interferometer 500 to generate IR light with an in-phase wave and an out-of-phase wave for every wavelength generated by the IR light. In one embodiment, a light source 480 includes the interferometer 500 and the IR source 504 as discussed in further detail in reference to FIG. 5B. A HeNe laser may be utilized as a clock to track the modulation of the interferometer 500. The in-phase and out-of-phase IR light waves may then be transmitted through the sample in a sample holder 462. In one embodiment, the sample holder 462 may include a wafer (e.g., chip) and/or a wafer holder. A read head 458 can be moved above (or below depending on the configuration of the system 400) and attach to the sample holder 462 to receive IR light transmissions that have been transmitted through the sample in the sample holder 462. The camera 448 can receive the optical signals from the read head and generate electrical signals that incorporate the IR absorption of the sample. The electrical signals can be sent to a computer 412 so a Fourier transform may be conducted to generate an IR absorption spectrum for each of the components in the sample.

FIG. 5A shows a detailed diagram of the multiple sample analyzing system 400 in accordance with one embodiment of the present invention. In one embodiment, the multiple sample analyzing system 400 includes the camera 448 which can receive optical signals. It should be appreciated that the camera 448 may be any suitable type of apparatus that can detect infrared light transmitted through the multiple samples to be analyzed as described above. The camera 448 may include an IR detector (e.g., focal plane array 488)(FPA)) that is enclosed within a dewar 450 to receive and record IR light. In one embodiment, the camera 448 may be configured to detect light wavelengths between about 5 to about 10 microns. In one particular embodiment, a 128×128 pixel HgCdTe focal point array (FPA) camera may be utilized. It should be appreciated that any suitable IR detecting/scanning device may be utilized in apparatuses described herein that can receive and record IR light such as, for example, scanning optics, rotating mirrors, single detector with movable mirror, etc.

The dewar 450 may be a jacket that can control the temperature of the IR detection environment. In one embodiment, the dewar 450 surrounds an optics 460 which can receive infrared signals that have passed through the sample desired to be examined. The temperature can be managed by application of temperature controlled fluid (e.g., nitrogen) in the jacket. The FPA 488 may then detect the IR light from the optics 460 and record data from such a detection.

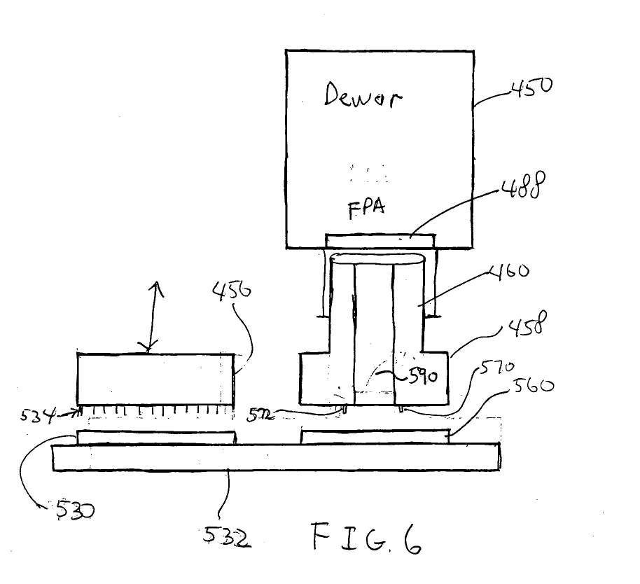

The multiple sample analyzing system 400 may also include a write head 456 and a read head 458. As discussed further below, the write head 456 may remove sample(s) from wells of a source plate and input the sample(s) into recesses (e.g., capillaries) in a wafer for IR spectroscopy. In one embodiment, the write head 456 is configured to move vertically onto and off of the source plate and the wafer. The read head 458 and the write head 456 being utilized with the source plate and the wafer is discussed in further detail in reference to FIG. 6.

To begin the testing, a source plate which contains samples to be tested may be moved under the write head 456. The source plate is discussed in further detail in reference to FIG. 15. The write head 456 can then move down onto the source plate to remove samples from the source plate. The write head 456 is then moved off of the source plate. Then the sample holder 462 can be moved under the write head 456 where the write head 456 may input the samples from the source plate into the sample holder 462.

In one embodiment of the sample testing, the sample holder 462 may be located within an active area 454 that is a region within the system 400 that has a controlled nitrogen gas atmosphere so the analysis environment is kept in a substantially constant state. The sample holder 462 may be located on a movable table that moves the sample holder below either a write head 456 or a read head 458. It should be appreciated that other embodiments may be utilized where the camera 448, read head 458, and/or write head 456 are located below the sample holder 462. In addition, the movable table (as discussed further in reference to FIG. 6) may also move a source plate with a plurality of samples to be analyzed under the write head 456 so the write head can withdraw the samples from the source plate and input the samples to the sample holder 462. After the samples have been inputted into the sample holder 462, the sample holder 462 may be moved under the read head 458.

In one embodiment, after the write head 456 has loaded the samples into the sample holder 462, the sample holder 462 may be moved under the read head 458. The read head 458 is configured to move vertically onto the wafer which contains the sample(s) to be analyzed. Then the read head 458 may move down onto the sample holder 462. In one embodiment, the read head 458 attaches to the sample holder 462 and the light source 480 may transmit the IR light through the sample holder 462. Therefore, in one embodiment, the read and write heads 458 and 456 respectively may be movable vertically so when the sample holder 462 is moved below either of the read and write heads 456 and 458, either one of the read and write heads 456 and 458 may move down over and/or onto the sample holder 462.

The read head 458 may also include a plurality of probes (e.g., voltage pins) which can apply an electrical charge to the two ends of each of the capillaries defined on the wafer. The read head 458 may therefore be a voltage applicator. The application of the electrical charge can facilitate isoelectric focusing to separate biological molecules. The read head 458 may also have a window that is transparent to IR light so the IR light transmitted from below the sample holder 462 can be transmitted through the window of the read head 458 to be detected by the FPA 488 of the camera 448. The read head 458 and the sample holder 462 are discussed in further detail in reference to the Figures discussed below.

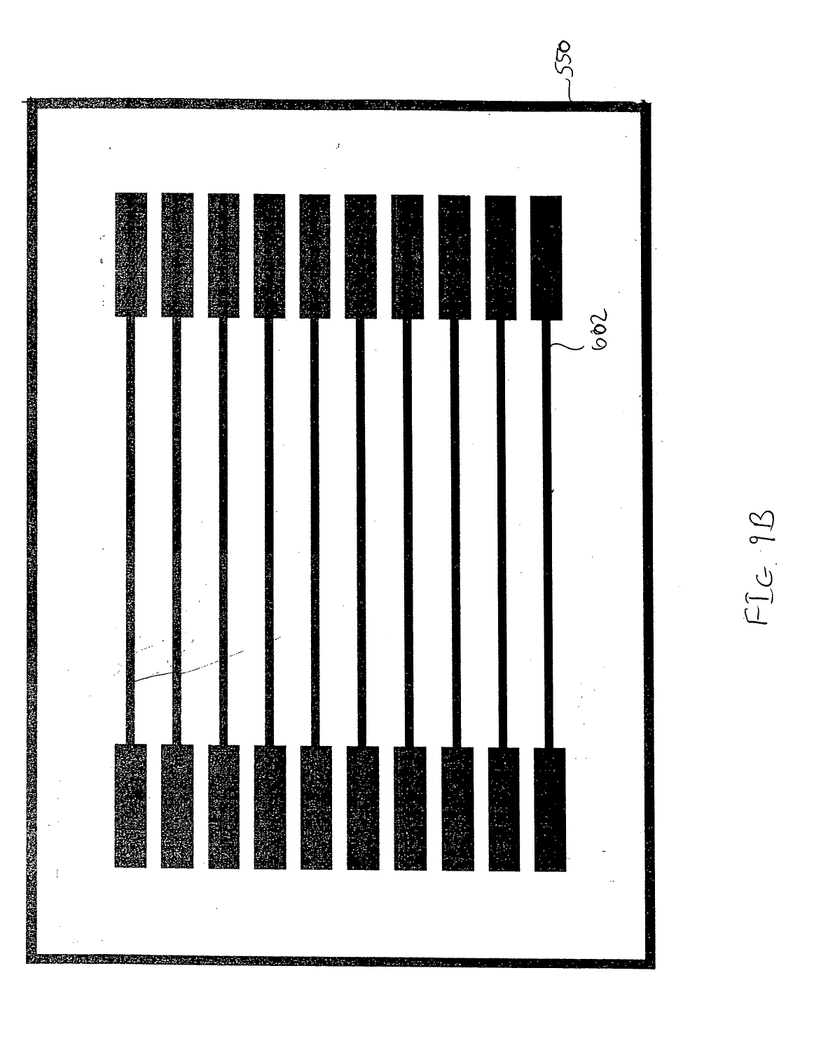

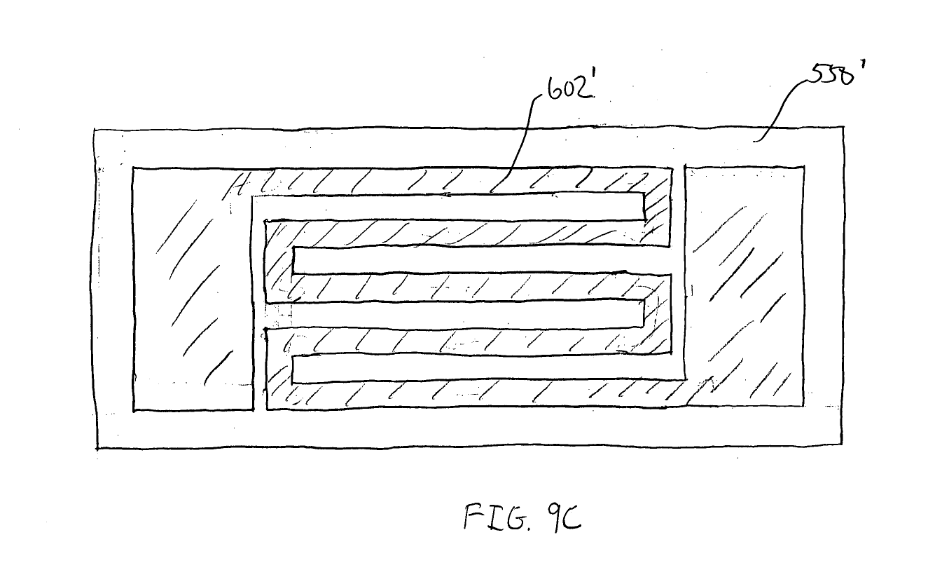

In one embodiment, the light source 480 may be located within the multiple sample analyzing system such that infrared light can be applied to a sample contained within the sample holder 462. The light source 480 may include the interferometer as discussed in further detail in reference to FIG. 5B. In one embodiment, the sample holder 462 may be a substrate with multiple recesses such as, for example, capillaries defined therein where each of the recesses is configured to contain a sample to be analyzed. In another embodiment, the sample holder 462 may include a wafer attached to a wafer holder. The recesses that are defined in the wafer are discussed in further detail in reference to FIGS. 9A-9C and 10A.

In operation, biological components within the sample may absorb certain wavelengths/frequencies of IR light depending on the biological composition of the components. In one embodiment, the IR light that has been transmitted through the sample holder 462 is detected by an FPA 488 of the camera 448. A window located at the end of the dewar 450 that is transparent to IR light can allow IR light to be detected by the FPA 488. The optical signal received by the FPA 488 can be transmitted to electronics 452 located within the dewar 450. As known to those skilled in the art, the dewar 450 may include the electronics 452 which can assist in managing the focal plane array by controlling the frame rate, clock cycle, etc. The electronics 452 may also facilitate communication between the camera 448 and a frame grabber 444 within a computer 412. Therefore, the optical signal can be transmitted from the dewar 450 to the frame grabber 444 and stored within a memory 446. The memory 446 within the computer 412 may be a cache memory which can receive and store data from the frame grabber 444. By utilization of the memory 446 such as, for example, the cache memory, use of a high frame rate in the IR spectroscopy process can be enabled.

The processor 442 can run a program 440 which may be configured to manage the light source 480 and the camera 448 to transmit through sample(s) and detect the optical signals that have been transmitted through the sample(s). The optical signal received from the camera 448 may be used to determine/characterize the composition of the sample(s) within the sample holder 462.

FIG. 5B illustrates an interferometer 500 in accordance with one embodiment of the present invention. In one embodiment, the interferometer 500 may be the light source 480 as shown above in reference to FIG. 5A. The interferometer may include an infrared (IR) source 504 that can generate IR light. It should be appreciated that the IR source 504 may be configured to generate beams of light waves that are in the infrared spectrum. In one simplified example of the interferometer 500 in operation, IR light beams 514 and 516 are shown as being generated by the IR source 504. It should be appreciated that having two IR light beams 514 and 516 are just an examples to show the workings of the interferometer; therefore, any suitable types and/or numbers of beams may be utilized herein. Consequently any suitable type of IR light may be generated by the IR source and processed by the interferometer 500 to generate in-phase IR light waves and corresponding out-of-phase IR light waves.

The light beam 514 can be reflected off of a mirror 515 toward a beam splitter 510. The light beam 514 reflected off of the mirror 515 is shown as light beam 514-1. A portion of the light beam 514-1 reflects off of the beam splitter 510 and forms light beam 514-2. Another portion of the light beam 514-1 does not reflect off of the beam splitter 510 and moves through the beam splitter 510 and forms light beam 514-4. The light beam 514-2 reflects off of the mirror 508 and forms light beam 514-3 which is one type of light transmitted to the sample. The light beam 514-4 reflects off of a mirror 512 which generates light beam 514-5. Light beam 514-5 reflects off of the beam splitter 510 and forms light beam 514-6 which is configured to be out of phase with the light beam 514-3 because of the different distances traveled by the lights. The mirror 508 may be moved to different distances away from the beam splitter 510 to generate the differing distances that the two split light beams travel. By having the split light beams travel different distances, one beam that is in phase and another light beam out of phase may be generated.

The light beam 516 can be reflected off of a mirror 515 toward the beam splitter 510. The light reflected off of the mirror 515 is shown as light beam 516-1. A portion of the light beam 516-1 reflects off of the beam splitter 510 and forms light beam 516-2. Another portion of the light beam 516-1 does not reflect off of the beam splitter 510 and moves through the beam splitter 510 and forms light beam 516-4. The light beam 516-2 reflects off of the mirror 508 and forms light beam 516-3 which is one type of light transmitted to the sample. As discussed above, the mirror 508 may be moved different distances away from the beam splitter 510 so the light beams split by splitter 510 may travel different distances. The light beam 516-4 reflects off of a mirror 512 which generates light beam 516-5. Light beam 516-5 reflects off of the beam splitter 510 and forms light 516-6 which is configured to be out of phase with the light beam 516-3 because of the different distances traveled by the lights. In such a manner, the interferometer is configured to generate infrared light with infrared light waves that may be out-of-phase.

The light source 480 may include a laser 501 which can set the modulation for the interferometer. It should be appreciated that any suitable device may be used to modulate the light from the laser 501 such as, for example, an encoder with a motor to track a position of the moving mirror used to differentiate the passage distance for in-phase and out-of-phase IR light waves. The light generated by the laser 501 may be transmitted to the beam splitter 510 which may split the laser light as with the light beams 514 and 516. A laser detector 518 may be configured to detect the light from the laser 501 so the laser 501 may be used as a reference light for managing the phase shifting of the lights 514 and 516.

FIG. 6 shows a read head 458 and a write head 456 in accordance with one embodiment of the present invention. In one embodiment, a source plate 530 may be located on a table 532 that can move laterally in any suitable direction to move the source plate 530 below the write head 456. It should be appreciated that the table 532 may be configured to move in any suitable direction (vertically, horizontally, etc.) depending on the configuration of the system. In one embodiment, the write head 456 is configured to include pins 534 that can remove samples from a plurality of wells in the source plate 530. In one embodiment, every three wells may correspond to a single capillary in the wafer 550. It should be appreciated that the write head 456 may utilize any suitable apparatus to remove samples from the source plate 530 and input the samples to the sample holder 462 such as, for example, using pins, tubes, etc. In one embodiment, the write head 456 can use pressure differences as generated by the pins 534 to remove the samples and input the samples. In one embodiment, biological samples in fluid may be removed through an internal passage defined in the pins 534 and the biological samples may be inputted into the sample holder 462 from the internal passage defined in the pins 534.

It should be appreciated that the write head 456 can include any suitable number of fluid removal implements (e.g., pins) such as, for example, 1, 20, 50, 100, etc. depending on the number of samples desired to be transported to the sample holder 462. In one embodiment, the write head 456 may have 30 pins. It should also be appreciated that the pins 534 may be in any suitable configuration as long as the configuration of pins enable removal of samples from the source plate 530 and input of samples to the sample holder 462 in an intelligent manner. In one embodiment, the pin configuration in a 30 pin write head may have 3 columns and 10 rows of pins. In such a configuration, each row of pins can input fluids into a single capillary in a 10 capillary sample holder as described in further detail in reference to FIGS. 9, 10A, and 10B.

The read head 458 may be coupled to the camera and can be maneuvered up and down to connect to the sample holder 462 when, in one embodiment, the sample holder 462 is moved into position directly underneath the read head 458. The read head 458 may include the window 590 through which the IR light that has been transmitted through the sample(s) can be detected by the focal plane array 488. In one embodiment, the focal plane array 488 may be included inside the dewar 450 so the conditions for IR light detection can be controlled.

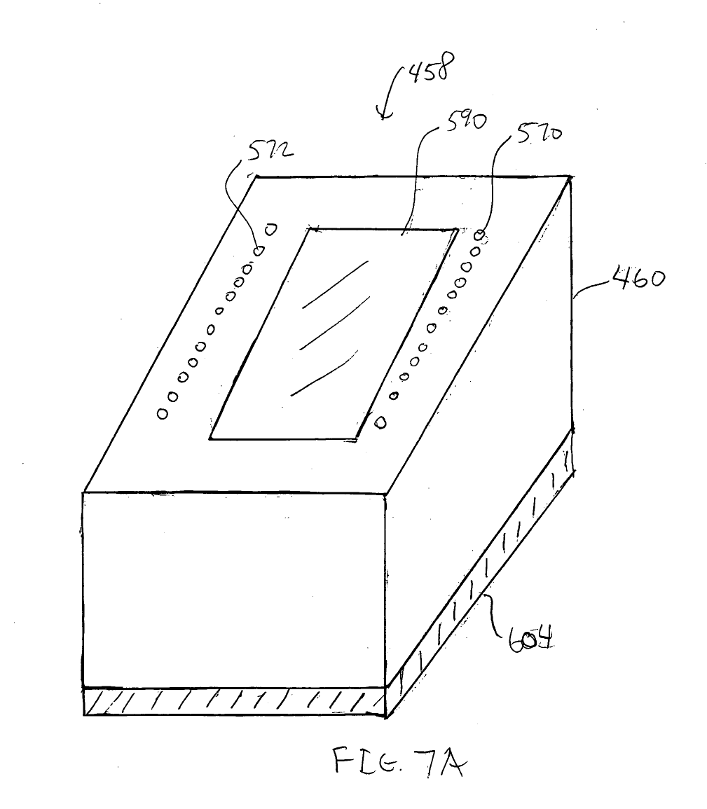

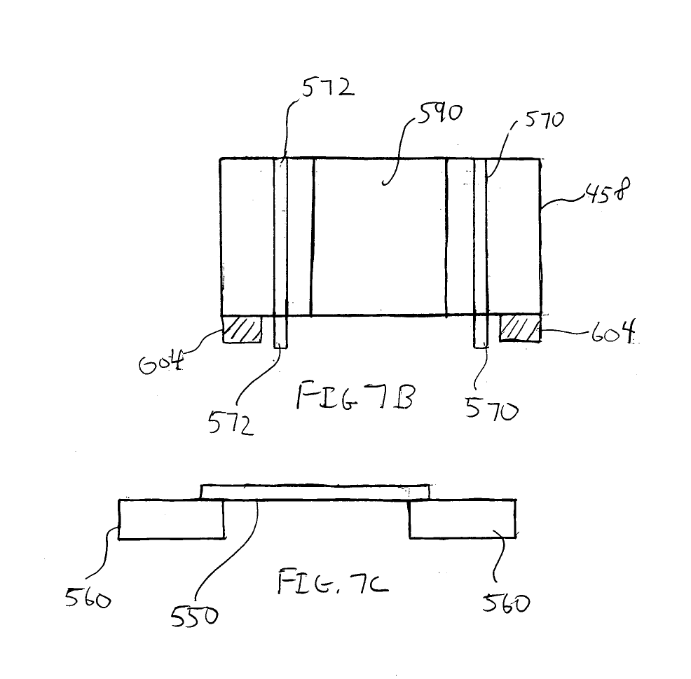

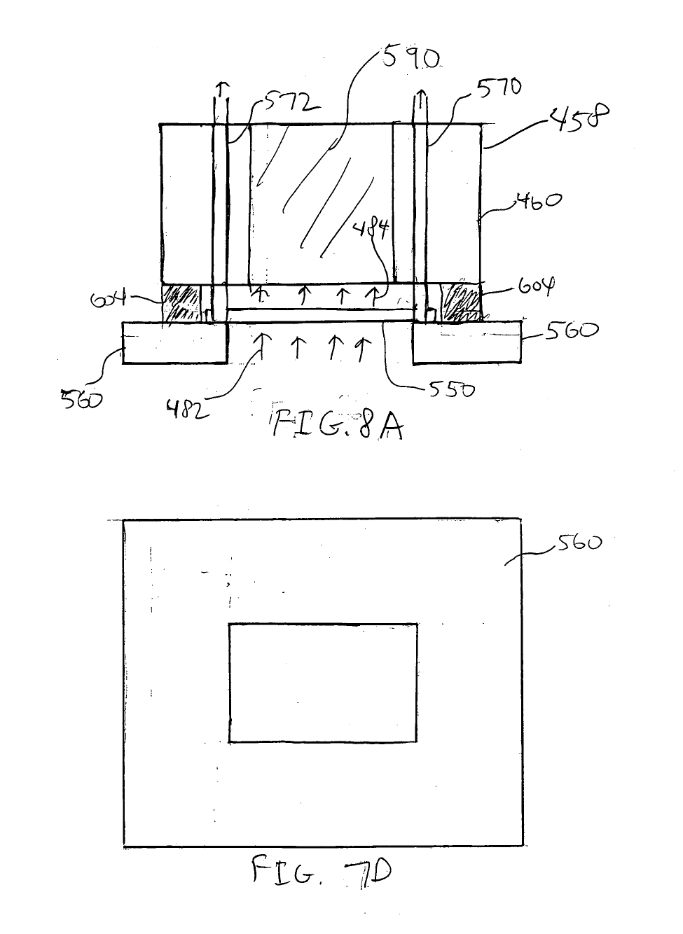

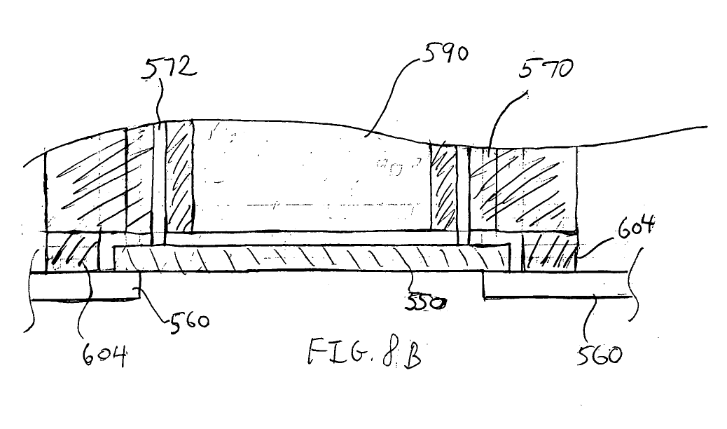

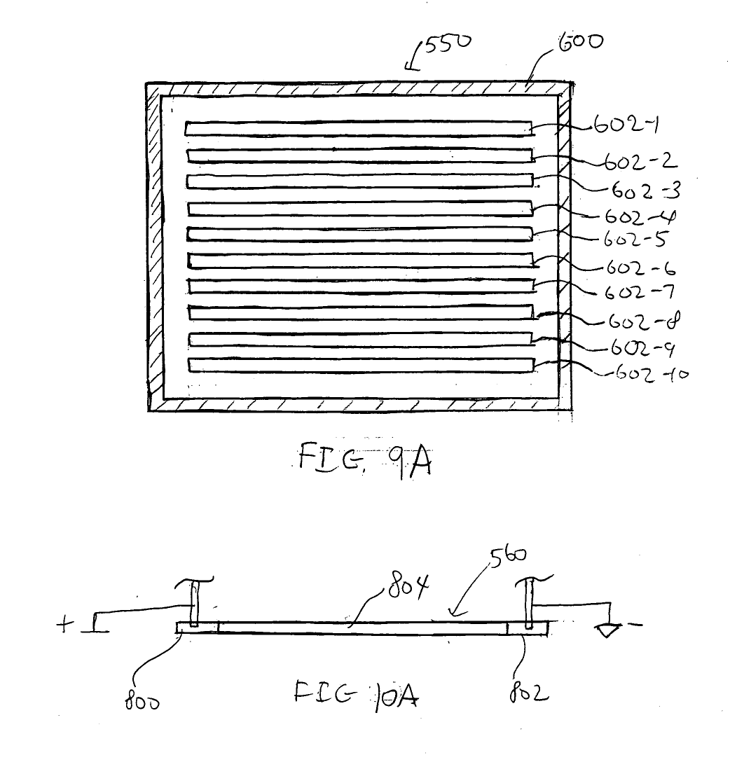

FIG. 7A shows a read head 458 in accordance with one embodiment of the present invention. The body of the read head 458 may be made from any suitable material such as, for example, plastic. In addition, the read head 458 may be any suitable size and shape as long as the read head 458 can effectively receive IR light transmitted through the samples. In one embodiment, the read head 458 is about 3 mm in height and is configured to attach to the sample holder 462. The read head 458 may include the window 590 which can correspond in size and shape to a portion of the wafer where the sample(s) is contained. The window 590 may be any suitable material that is substantially transparent to IR light. The read head 456 may also include a plurality of voltage pins 570 and 572. In one embodiment, a single set of voltage pins 570 and 572 exists for every recess where the sample may be held (e.g., capillary) in the wafer. Therefore, depending on the size and shape of recesses, the location and number of voltage pins 570 and 572 may change. In addition, depending on the layout of the recesses in the wafer, the size and shape of the window 590 may be differ. Also, the read head 458 may include a gasket 604 that substantially surrounds the window 590 and which can seal the read head 458 to the wafer holder 560 as shown in FIG. 8A. Once the read head 458 is sealed on the wafer holder 560, the samples within the capillaries are sealed from the atmosphere thereby substantially reducing premature evaporation. Because in one embodiment, the capillaries contain small amounts of samples, the reduction of evaporation greatly increases the time available for sample testing.

FIG. 7B depicts a side view of the read head 458 in accordance with one embodiment of the present invention. In one embodiment, the pins 570 and 572 extend out of the read head 458 so when the read head 458 is attached to the sample holder 462, the pin 570 dips into one end of a particular capillary and the pin 572 dips into the other end of the particular capillary. It should be appreciated there may be any suitable number of pins 570 and 572 on the read head depending on the number of capillaries to be examined. In one embodiment, for each capillary on the sample holder, one pin 570 and one pin 572 may be utilized.

FIG. 7C illustrates a side view of a wafer 550 attached to a wafer holder 560 in accordance with one embodiment of the present invention. In one embodiment, the wafer holder 560 may be configured to hold the wafer 550 around an edge portion of the wafer 550. In such a configuration, an opening in the middle of the wafer holder 560 enables IR light to be transmitted directly to the wafer 550 through the opening. One embodiment of the wafer holder 560 is described in further detail in reference to FIG. 7D.

FIG. 7D illustrates a top view of the wafer holder 560 in accordance with one embodiment of the present invention. It should be appreciated that the wafer holder 560 may be any suitable size and/or shape as long as the wafer 550 may be held and IR light can be transmitted through an opening of the wafer holder 560. In another embodiment, the wafer holder 560 may not have a opening as long as the wafer holder 560 is made from a material that is transparent to IR light. In one embodiment, the holder 560 may rectangular in shape with an opening in the middle so light can be transmitted into one side of the wafer 550 and out of the other side of the wafer 550. It should also be appreciated that the wafer holder 560 may be made out of any suitable material as long as the wafer 550 may be held securely.

FIG. 8A shows a cross-sectional view of the read head 458 attached to the sample holder 462 in accordance with one embodiment of the present invention. In one embodiment, the sample holder 462 includes a wafer 550 with recesses (e.g., capillaries) defined therein attached to the wafer holder 560. The wafer 550 is described in further detail in reference to FIGS. 9A and 9B. The wafer 550 may be attached to the wafer holder 560 so that the capillaries defined in the wafer 550 are located over an opening of the wafer holder 560. Therefore, when the wafer 550 is made from a material that is transparent to IR light, the IR light may be transmitted from below the wafer holder 560 through the wafer 550 into a window 590 in the read head 458. As discussed above, IR transparent materials may be any suitable material that can be substantially transparent to a portion or all of the IR light spectrum. In addition, the wafer holder 560 may alternatively not have an opening as long as the material from which the wafer holder 560 is constructed is substantially transparent to IR light.

FIG. 8B illustrates a close-up view of the read head 456 connecting with the wafer holder 560 in accordance with one embodiment of the present invention. In one embodiment, the read head 456 includes a gasket 604 that attaches to a surface of the wafer holder 560. It should be appreciated that the gasket 604 may be made from any suitable material that can substantially seal the read head 456 to the wafer holder 560 such as, for example, rubber, elastomers, etc. The read head 456 includes the window 590 through which IR light transmitted through the wafer 550 can enter. The read head 456 also includes voltage pins 570 and 572. The voltage pins 570 and 572 may be applied to the capillaries in the wafer 550 so an electric field can be applied across the length of the capillaries so isoelectric focusing may be conducted.

FIG. 9A shows a top view of the wafer 550 that is configured to include recesses where samples can be inputted and analyzed in accordance with one embodiment of the present invention. In one embodiment, the wafer 550 may have any suitable number and/or type of recess(es) (e.g., capillaries) defined in the wafer 550 to hold samples to be tested. In one embodiment, the recesses are a plurality of capillaries 602-1 to 602-10 that may be spaced parallel to each other. Other exemplary forms of recesses that may be defined on a surface of the wafer 550 is described in further detail in reference to FIGS. 10C through 10E. In one embodiment, the wafer 550 and/or wafer holder 560 may form a bottom portion and the read head 458 may form a top portion in a connected structure. Therefore, when the read head 458 attaches to the wafer holder 560, the capillaries may be sealed by the read head 458 so the samples are not exposed to the outside environment for an extended period of time. This may reduce evaporation of the sample in a significant manner. In yet another embodiment, the wafer 550 may include containment spaces that are entirely defined within the wafer 550 thereby reducing evaporation of the samples.

FIG. 9B illustrates a top view of an alternative wafer 550 in accordance with one embodiment of the present invention. In one embodiment, the wafer 550 may include a plurality of recesses (e.g., capillaries) that are of the type as discussed in further detail in reference to FIG. 13. As discussed in FIG. 13, the capillary 602 has a first end and a second end that are each larger in width than the middle portion of the capillary 602. In such a configuration, voltage probes may be applied to the first end and the second end while the sample may be located in the middle portion.

FIG. 9C shows a top view of a wafer 550′ with extended length capillaries 602′ in accordance with one embodiment of the present invention. In this embodiment, a length through which the components of a biological sample may travel is extended by generating a substantially overlapping capillary configuration. In one embodiment, the capillary 602′ may have any suitable size as described above and in a preferable embodiment, the capillary may be about 75 microns in width. In one embodiment, depending on the travel distance desired, extra cycle(s) of turns in the capillary may be incorporated thereby increasing the distance that the components have to travel. In such an embodiment, a larger pH gradient may be used and a lower intensity electrical field may be utilized. It should be appreciated that any suitable intensity of electrical field as described above may be utilized, and in a preferable embodiment, an electrical field of about 20 V/cm may be utilized. In one exemplary embodiment, a fluid sample may be inputted in a midpoint of the capillary between the anode and the cathode. Therefore, by increasing the effective length of the capillary, the effective resistance to movement may be increased and a lower intensity of electrical field may be utilized to separate the components of the biological sample.

FIG. 10A shows a side view of the capillary 602 in accordance with one embodiment of the present invention. The capillary 602 may be configured so components within a sample may be separated. In one embodiment, isoelectric focusing may be utilized for molecular separation. In another embodiment, electrophoresis may be used for molecular separation. It should be appreciated that the description of isoelectric focusing above is one exemplary separation technique that may be utilized and other suitable types of molecular separation techniques may be utilized.

In one embodiment, the capillary 602 has three sections. A first section may be a probe region 800, a complimentary probe region 802, and a sample receiving region 804. The probe region 800 of the capillary 602 may configured to hold an acidic solution and the complimentary probe region 802 may be configured to have a basic solution (or vice versa depending on which region has a negative or a positive charge) while sample receiving region 804 is configured to receive and hold the sample that is to be analyzed. In one embodiment, a pH gradient is generated between one end of the capillary 602 and the other end of the capillary 602. In addition, a voltage is applied across the length of the capillary 602 to generate an electrical field so depending on the electrical properties of the molecules in the sample, different components of the sample move to different regions of the capillary. In one embodiment, a voltage of between about 20 V to about 200 V is applied. To put it a different way, an electrical field that may be generated along the capillary may be between about 100 V/cm and 300 V/cm. In a preferable embodiment, a voltage of about 100V may be applied.

Therefore, by applying both a pH gradient and an electric field, different regions of the capillary 602 can have different electrical and acidic levels. Components being analyzed such as, for example, proteins may have different electrical charges. Consequently, due to different isoelectric properties of different biological/chemical components, each particular component of a sample may move to different regions of the capillary 602. During movement along the capillary, the components may move along the pH gradient and gain or lose protons during depending on the location of the component along the pH gradient. Once the component moves to a location where the component is uncharged, the movement may stop. By using this methodology certain components (e.g. proteins, protein interaction resultant, amino acids, genetic material, etc.) within a sample being analyzed can be separated for further analysis by IR spectroscopy.

FIG. 10B illustrates the write head 456 inputting a fluid sample 818 into the capillary 602 in accordance with one embodiment of the present invention. It should be appreciated that the fluid may be any suitable type of sample such as, for example, proteins, protein interaction resultant, genetic material, amino acids, etc. In one embodiment, the write head 456 and the read head 458 are shown as being above the wafer 550 with the write head in position to input a first probe fluid 816 from pin 534a, the fluid sample 818 from pin 534b, and the second probing fluid 820 from pin 534c into the capillary 602. The first probe fluid 816 may be inputted into the probe region 800, the fluid sample 818 may be inputted into the sample receiving region 804, and the second probe fluid 820 may be inputted into the complimentary probe region 802. In one embodiment, the first probe fluid may be any suitable acidic fluid (e.g., phosphoric acid (H₃PO₄)) and the second probe fluid may be any suitable basic fluid (e.g., sodium hydroxide (NaOH)) In another embodiment, if electrophoresis is utilized to separate the biological components within the biological sample, potassium chloride may be utilized.

In one embodiment, the regions 800, 802, and 804 are recesses on an active surface 806 of the wafer 560. In one embodiment, the active surface 806 is on an opposite side as a backside surface 808. As discussed in more detail in reference to FIG. 14, the recess making up the regions 800, 802, and 804 may be defined on the active surface 806 by etching the active surface 806. It should be appreciated that any suitable etching operation as known to those skilled in the art may be utilized.

It should be appreciated that any one, combination of, or all of the capillary 602, pins 562, and voltage pins 570 and 572 may be coated or made from any suitable material that reduces attraction to the sample(s). In one embodiment, the pins 562 may be coated with a material such that the sample(s) are not attracted to the pins 562. In another embodiment, the voltage pins 570 and 572 may be coated with a material that is non-reactive with the sample(s). In another one embodiment, the recesses such as, for example, the capillary 602 may be coated with a material such that surface charge on the surface of the capillary 602 may be reduced.

FIGS. 10C through 10E illustrate recessed regions that can be substituted for the capillaries 602 as discussed herein to contain the sample for analysis. As shown in the FIGS. 10C through 10E below, the recessed region for holding the sample may be any suitable size or shape. It should also be appreciated that although only one recessed region is shown on the wafer, any suitable numbers of recessed regions may be defined on the wafer.

FIG. 10C depicts an oval shaped recessed region 811 in accordance with one embodiment of the present invention. In one embodiment, the oval shaped recessed region 811 has the probe region 800, the sample receiving region 804, and the complimentary probe region 802.

FIG. 10D illustrates a square shaped recessed region 812 in accordance with one embodiment of the present invention. In one embodiment, the oval shaped recessed region 812 has the probe region 800, the sample receiving region 804, and the complimentary probe region 802.

FIG. 10E shows a round shaped recessed region 814 in accordance with one embodiment of the present invention. In one embodiment, the oval shaped recessed region 814 has the probe region 800, the sample receiving region 804, and the complimentary probe region 802.

FIG. 11 illustrates an imaging process that reveals molecular details such as location, movement, and binding of solutes from sample 810 introduced to an isoelectric separation chamber 820 in accordance with one embodiment of the present invention. In one embodiment, the isoelectric separation chamber may be the capillary 602 defined in the wafer 550. Bands 825 may form in the chamber 820 by isoelectric focusing. Infrared optics and detector 830 simultaneous image bands 825 to generate signal patterns 840. The signal patterns are used to determine spectral changes that occur in time as depicted by graph 850. The ability to carry out hyperspectral measurements in real time allow new types of isoelectric focusing that do not rely on high density, viscous or gel like matrices. For example, a complex two dimensional pattern can be established, in a bull's eye conformation with annular rings around a center electrode for assay of multiple samples.

The system may be combined with a counter current flow of solute, binding partner, or substrate that may be constantly replenished or expose a focused sample to a periodic or other varying concentration to determine the effect of other substances including enzyme substrates on conformational spectra. This embodiment is particularly useful for drug discovery in instances where a test compound is consumed during reaction with an enzymatic molecule or macro molecular complex.

FIG. 12A depicts a sample that has been analyzed through IR spectroscopy in accordance with one embodiment of the present invention. In one embodiment, after the sample has been inputted into the capillary 602, a pH gradient is generated as described above in reference to FIG. 10. A voltage may be applied between the two ends of the capillary 602 so different molecules of the sample move to locations in the pH gradient where the molecule is electrical equilibrium. Bands 842 and 844 in this exemplary process shows the location where two different components of the sample have an electrical charge of substantially zero. Therefore, different chemical/biological molecules in the sample may be separated using this type of methodology.

Once the separation has taken place, IR spectroscopy as described herein can be conducted on the molecules in the bands 842 and 844 of the capillary 602 to obtain the IR light absorption spectrum for each of the bands 842 and 844 of the capillary 602. Therefore, by using both isoelectric focusing and IR spectroscopy, different molecules within a sample may be identified in an intelligent and cost-effective manner. Moreover, by having multiple capillaries defined in the wafer 550, a large number of samples may be concurrently analyzed. By using this methodology, the testing conditions may be made substantially identical between the capillaries thereby substantially reducing testing errors that may be introduced by change in testing conditions from one test to another test.

FIG. 12B shows a close-up view of the IR light absorption spectrum for a particular sample in accordance with one embodiment of the present invention. As shown in FIG. 12B, each band shown in the capillary may represent a different type of molecule with different electrical properties. Therefore, due to the pH gradient and the voltage applied on the ends of the capillary 602, each of the chemicals in the sample move to different portions of the capillary where electrical equilibrium is achieved. Each of the bands can generate an IR light absorption spectrum thereby enabling intelligent determination, identification, and/or characterization of the samples being tested.

FIG. 13 illustrates a top view of the capillary 602 in accordance with one embodiment of the present invention. In one embodiment, the capillary 602 has three regions 800, 802, and 804 as described in further detail above. In one embodiment, the probe region 800 is a cathode region where a positive charge is applied to the fluid in that region. In one embodiment, the sample receiving region 804 where the sample to be analyzed may be located. The complimentary probe region 802 is an anode region where the negative charge is applied in that region. The regions 800 and 802 may each hold a volume of fluid in a range from about 25 nl to about 50 nl. In a preferable embodiment, the regions 800 and 802 may each hold a volume about 25 nl. The region 804 may hold a sample fluid in a range from about 10 nl to about 100 nl and in a preferable embodiment, the region 804 may contain 15 nl of the sample fluid.

In one embodiment, each of the lengths 866 and 862 is between about 1 mm to about 3 mm and a distance 864 is between about 2 mm to about 10 mm. In a preferable embodiment, the lengths 866 and 862 may each be about 2 mm. A width 860 of the capillary 602, in one embodiment, is between about 50 microns to about 100 microns. In a preferable embodiment, the width 860 of the capillary 602 is about 125 microns. In one embodiment, widths 868 and 870 may each be between about 250 microns to about 1000 microns. The widths 868 and 870, in a preferable embodiment, are about 500 microns.

The capillary 602 may have any suitable depth depending on the desired volume of the capillary 602. In one embodiment, the capillary 602 may have a depth between about 5 microns to about 100 microns while in a preferable embodiment, the capillary is about 25 microns in depth.

FIG. 14 shows a side view of the wafer 550 in accordance with one embodiment of the present invention. As discussed above in reference to FIG. 13, the wafer 550 may include one or more of the capillaries 620 that may have a depth 900 between about 5 microns to about 100 microns. In a preferable embodiment, the depth 900 may be about 30 microns. In one embodiment, the capillaries may be defined on the surface of the wafer 550 by way of an etching process. Any number of etching techniques known to those skilled in the art may be utilized for the etching process. In one embodiment, a deep reactive ion etch (DRIE) may be utilized to generate the recesses on the surface of the wafer 550 to generate the capillaries 620. In one embodiment, the wafer 550 may be any suitable thickness and in a preferable embodiment, the wafer 550 may be between about 1 micron and 4 cm in thickness.

FIG. 15 depicts a source plate 530 in accordance with one embodiment of the present invention. In one embodiment, the source plate 530 includes a plurality of wells 952 that can contain any suitable fluid to be used in IR spectroscopy analysis. In one embodiment, for every three wells across each row, a first well is filled with a fluid that is to be inputted into an anode section of the capillary 460, a second well is filled with a sample to be analyzed, and a third well is filled with a fluid that is to be inputted into a cathode section of the capillary 460. In the exemplary embodiment shown in FIG. 15, source plate 530 includes 9 wells for every row. Therefore, three samples may be located in each row. It should be appreciated that the number of wells, the number of columns, and/or the number or rows in the source plate 950 may be any suitable number depending on the application desired. In addition, depending on the write head 456, any suitable shape of the wells and/or source plate 530 may be utilized. In one exemplary embodiment, a 1536 format as known to those skilled in the art may be utilized.

In one embodiment, the write head can move over the source plate 530 and the write head 456 can move down onto the source plate 950. The pins of the write head can draw and retain fluid from the wells 952 of the source plate 530. In one embodiment, the pins of the write head 458 may be configured so pins for the first three wells in a row are in line that dip into a top section 954 of the wells 952. The next three pins of the write head 458 for the second three wells in the row may be staggered so those pins dip into a middle section 956 of the wells 952. The last three pins of the row of the write head 456 may be staggered further so those pins dip into a bottom section 958 of the wells 952. In one embodiment, this type of pin configuration may be repeated for each set of pins configured to dip into a row of the wells 952 of the source plate 530.

The write head 456 can move up from the source plate 530. Then the source plate 530 may be moved out of the way and a sample holder with the wafer may be moved underneath the write head. The write head can move down onto the sample holder so the pins are placed in user defined locations in the capillaries to release the appropriate fluids. It should be appreciated that the write head 456 may utilize any suitable type of method and/or apparatus to remove fluid from the source plate 530 and to input the fluid into the sample holder such as, for example, pipetting, printing, syringe pumps, aspirating devices, etc. It should also be appreciated that the sample(s) may include any suitable type of additive that can manage surface tension of the sample(s). In one embodiment, additives for protein capillary isoelectric focusing may include detergents to prevent or limit precipitation such as, for example, Triton X-100, CHAPS, and octyl glucoside. In addition, urea can be added to suppress protein aggregation. In one embodiment, methylcellulose, polyvinyl alcohol, or other polymeric coatings reduce interactions with the capillary walls and prevent or reduce the electroendosmotic flow (EOF).

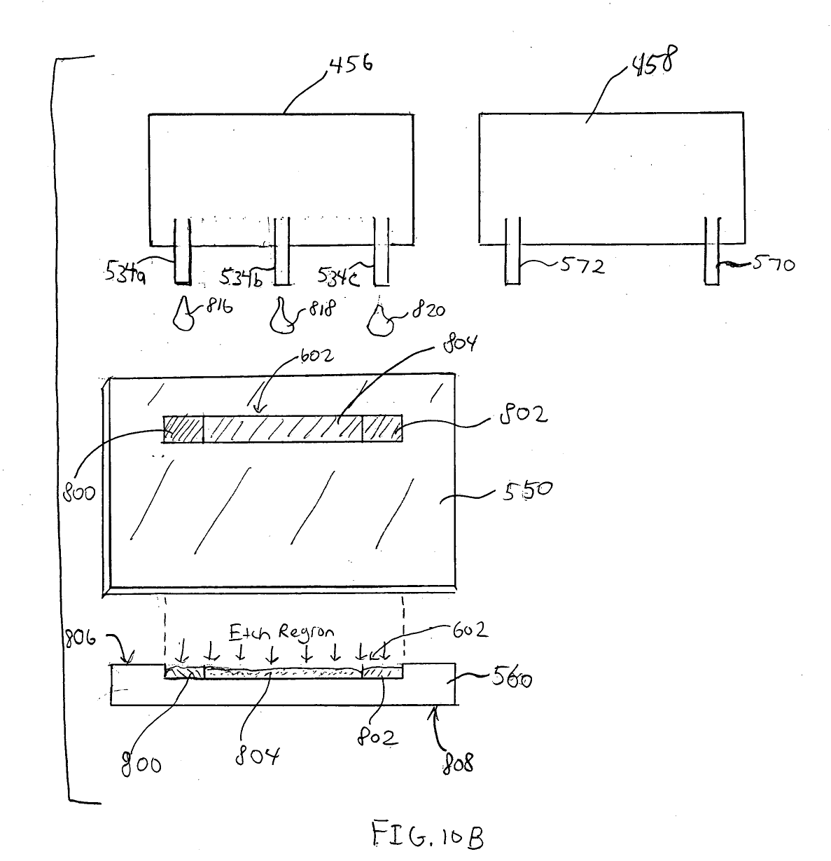

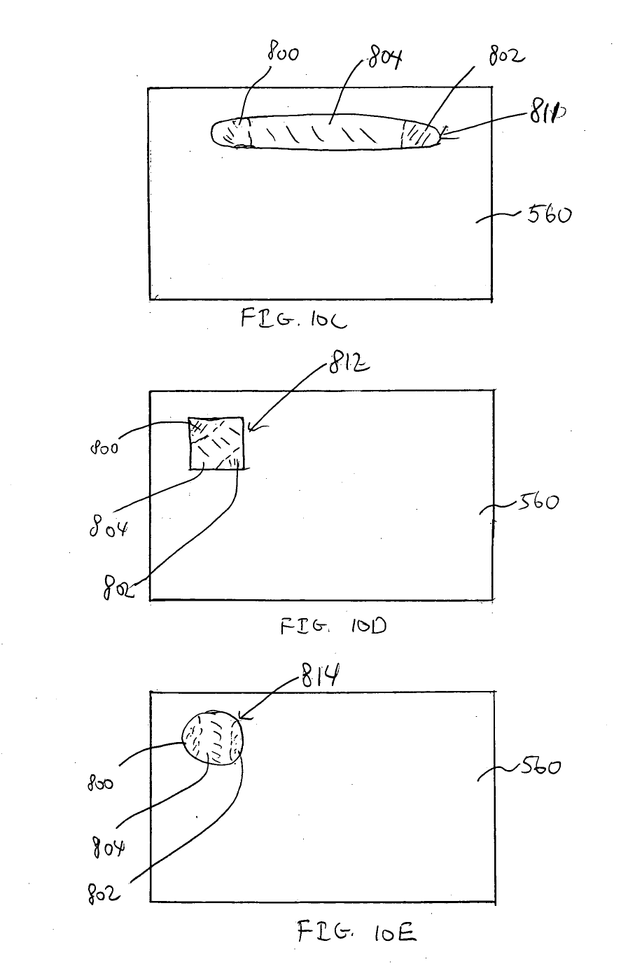

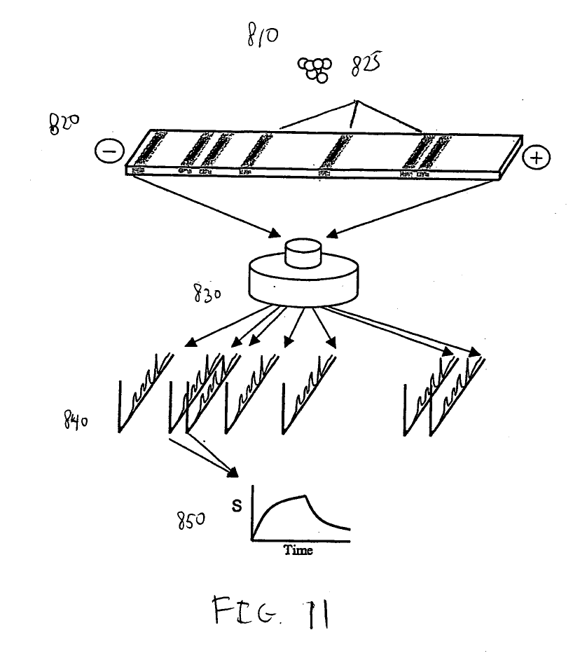

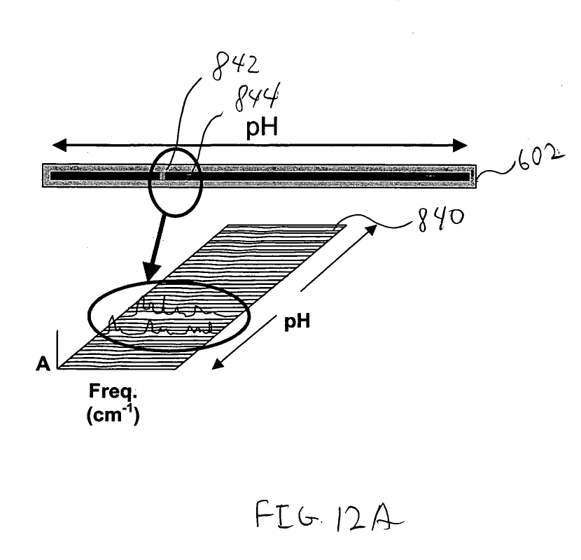

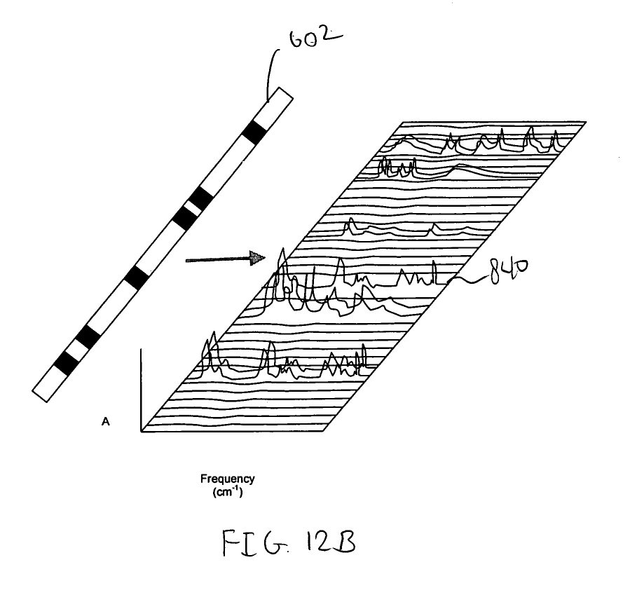

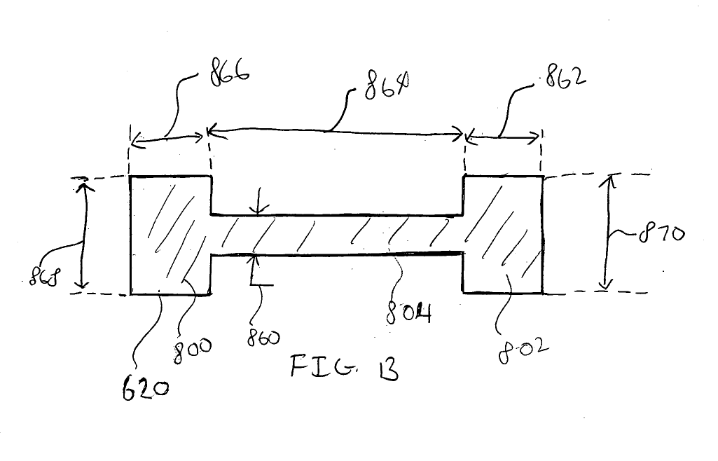

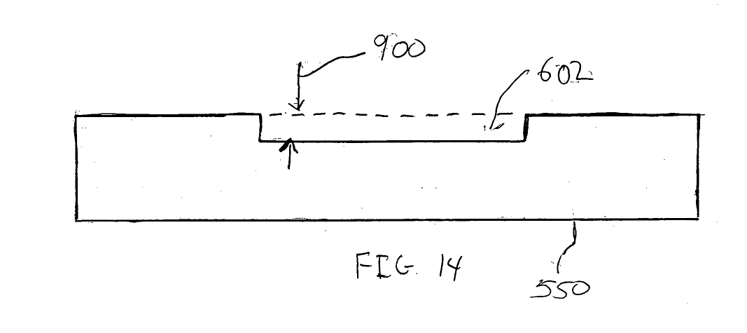

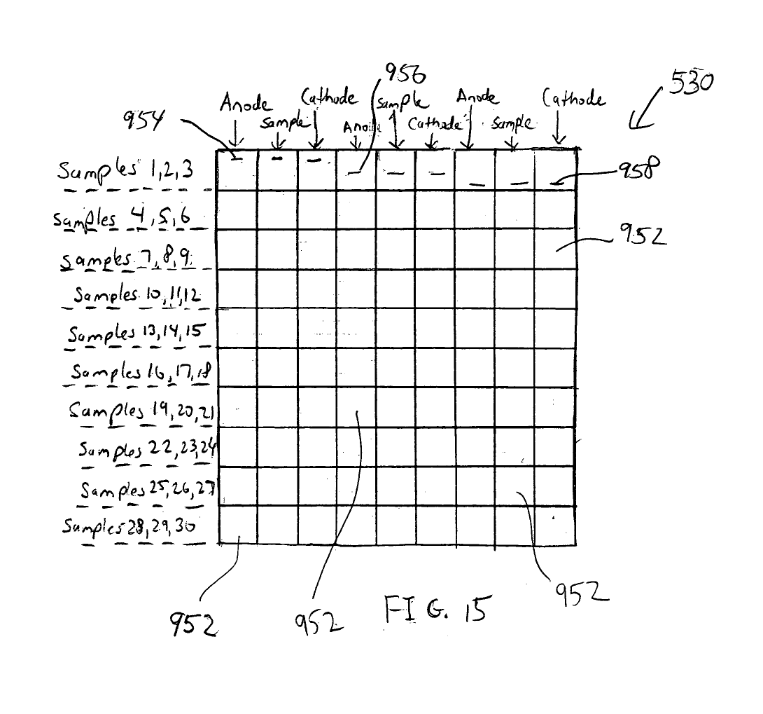

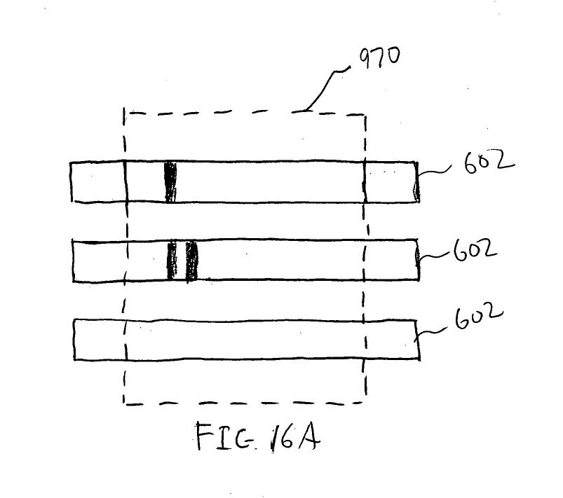

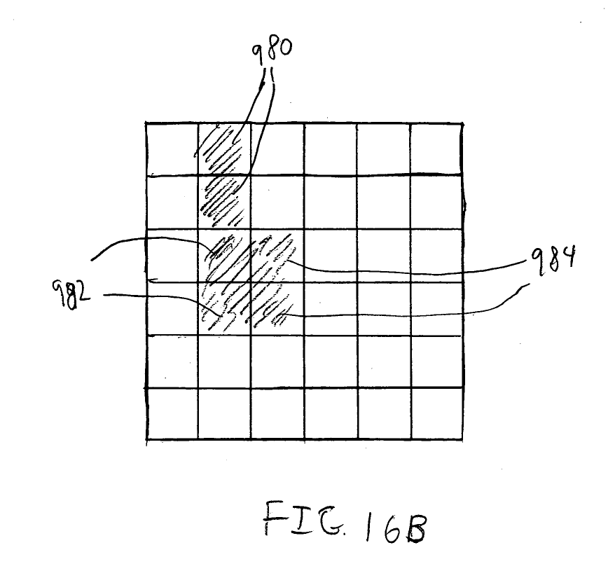

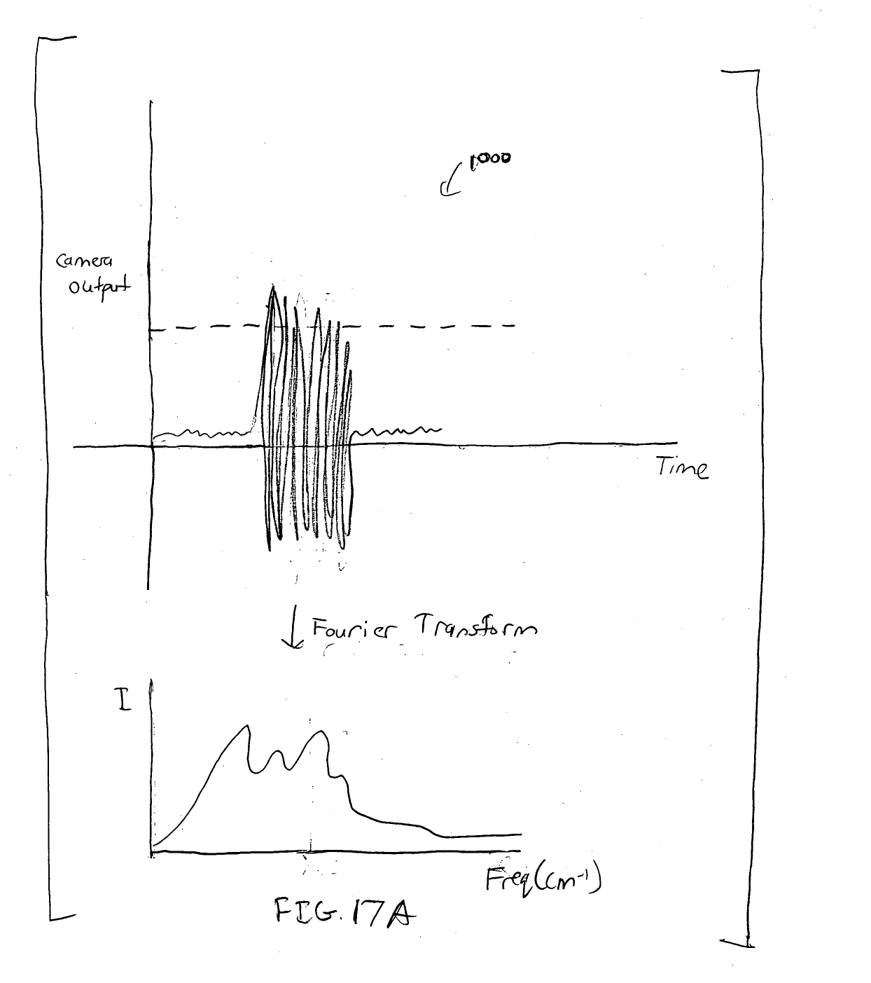

FIG. 16A shows a detection field 970 of the camera 448 in accordance with one embodiment of the present invention. The field 970 may be a region of space from which light may be detected. In one embodiment, the field 970 receives IR light transmitted through the sample receiving region 804 of the capillaries 602. As discussed in reference to FIGS. 10A and 10B, the sample receiving region 804 is the region of the capillary 602 where the sample to be analyzed is located. Therefore, any bands that may occur due to isoelectric focusing can have IR light applied to it and then have the IR absorption spectrum determined through the readings from the camera 448.

FIG. 16B illustrates an exemplary pixel pattern of a portion of the detection field 970 of the camera 448 in accordance with one embodiment of the present invention. The pixel pattern shown in FIG. 16B is a simplified view of a limited number of pixels that can detect IR absorption of the bands generated in an isoelectric focus operation. It should be appreciated that the pixel representation is simplified for purposes of explanation and that a much larger number of pixels may be utilized for IR light detection.

In the exemplary embodiment shown in FIG. 16B, pixels 980 receives IR absorption signals from a band on a first capillary as shown by the darkened pixels. The pixels 982 and 984 may receive IR absorption signals from two bands on a second capillary as shown by the darkened pixels. These bands correspond to the bands as shown in FIG. 16A.

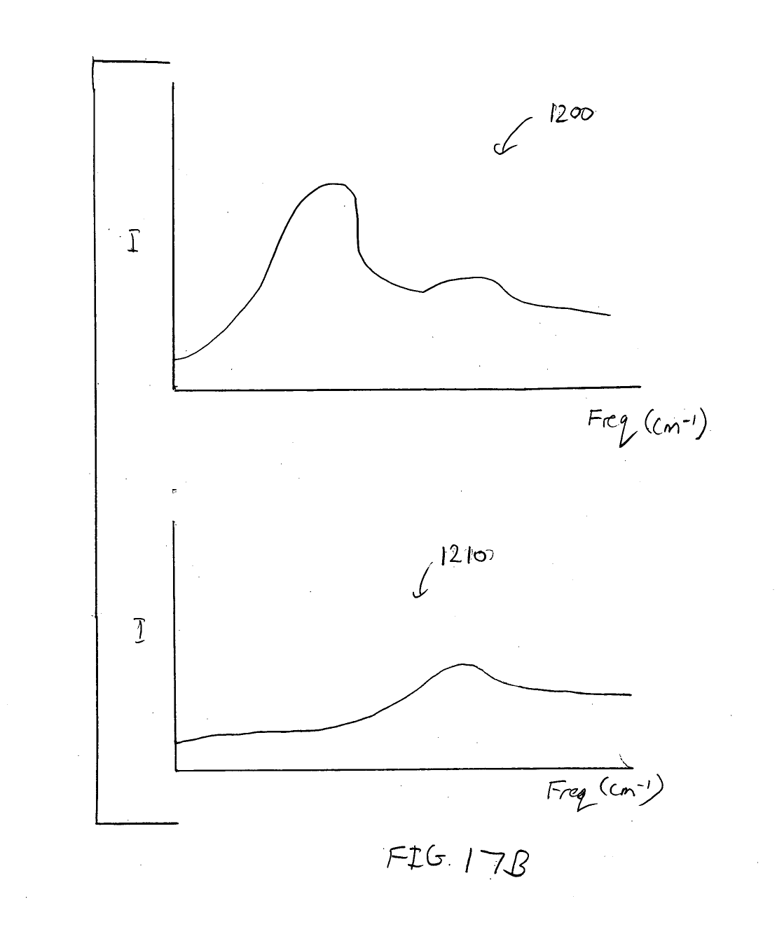

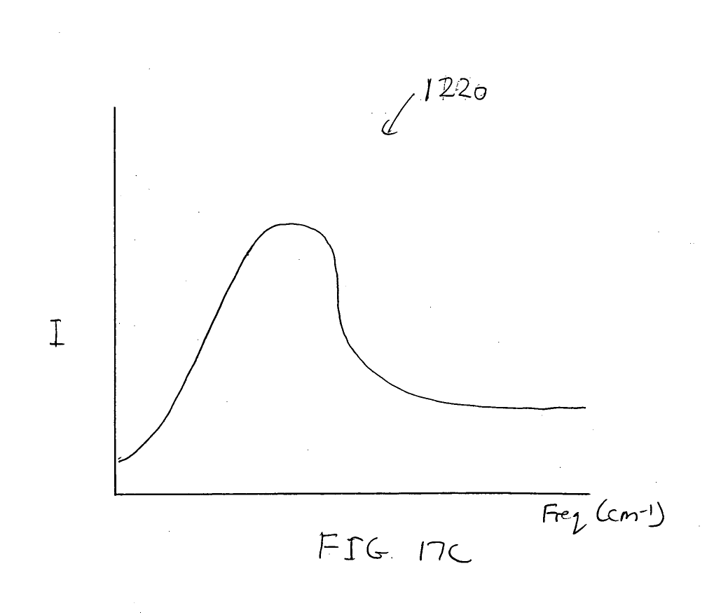

FIG. 17A illustrates a Fourier transform of data shown on a graph 1000 where camera output is plotted against time in accordance with one embodiment of the present invention. In one embodiment, the graph 1000 is generated which plots time on an x-axis and a camera output to a computer on a y-axis. The camera output has a very large amount of data points which can be processed by using an inverse Fourier transform. In one embodiment, any suitable type of Fourier Transform consistent with the methodology described herein may be utilized to generate an IR absorption spectrum where the x-axis represents a range of frequencies and the y-axis represents an intensity of the detected infrared light that was transmitted through a sample.

In one exemplary embodiment, an IR absorption spectrum for a biological sample before protein interaction may be generated and an IR absorption spectrum for a sample after protein interaction may be generated. After the two spectrums are generated, the common absorption regions can be canceled out and the remaining absorption spectrum can be utilized to determine the actual biological and/or chemical changes of a particular sample.

Numerous analyses may be conducted using the apparatus and method of the present invention. For example one protein may be analyzed for different reactivity with different drugs. In another example 10 different drugs may be tested with 10 different reactants. In yet another example, different concentrations of a same drug can be tested with a particular protein to determine effectiveness of a treatment with the drug. Therefore, the present invention may intelligent and powerful analyses of multiple biological samples.

FIG. 17B depicts graphs 1200 and 1210 that show an absorption spectrum of one band in a first capillary and a second capillary in accordance with one embodiment of the present invention. In one embodiment, the graph 1200 illustrates the IR absorption spectrum of a particular protein along with other fluid(s) typically utilized when conducting isoelectric focusing such as, for example, water, amphoteric small molecules, carrier ampholytes,. Graph 1210 illustrates the IR absorption spectrum of the fluid of graph 1200 without the protein. Therefore, graph 1210 shows a baseline IR absorption of the fluid(s) not including in the sample. As discussed in further detail in reference to FIG. 17C, both of the graphs 1200 and 1210 may be utilized to generate a new IR absorption spectrum to determine the identification of the sample.

FIG. 17C illustrates a graph 1220 that shows the IR absorption spectrum of only the sample as discussed in FIG. 17B in accordance with one embodiment of the present invention. In one embodiment, the graph 1220 is the difference of the IR absorption spectrum of graph 1210 of FIG. 17B subtracted from the IR absorption spectrum of graph 1200. Basically, all of the absorption peaks as shown in graph 1210 was removed from graph 1200. Because graph 1210 showed the IR absorption spectrum of the fluid(s) without the sample and graph 1200 showed the IR absorption spectrum with the same fluid(s) with the sample, the difference between the IR absorption spectrums of graphs 1210 and 1200 results in the IR absorption spectrum of just the sample.

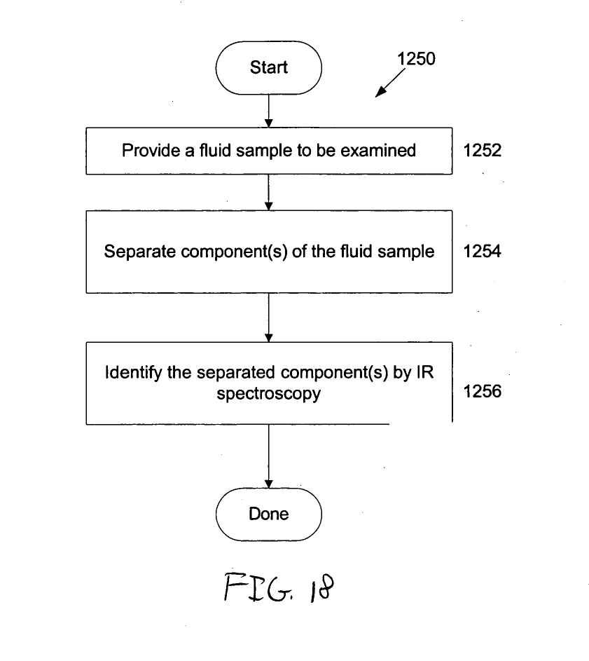

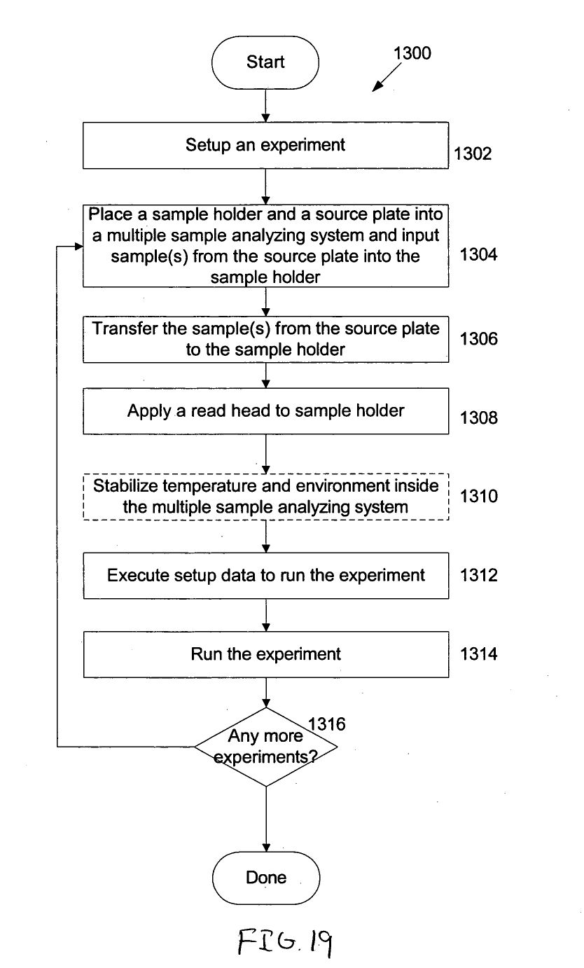

FIG. 18 shows a flowchart 1250 defining a method for examining a fluid sample in accordance with one embodiment of the present invention. It should be understood that the processes depicted in any of the methods and flowcharts described herein may be in a program instruction form written on any type of computer readable media. For instance, the program instructions can be in the form of software code developed using any suitable type of programming language. For completeness, the process flow of FIG. 18 will illustrate an exemplary process whereby a sample is analyzed through use of IR spectroscopy.

The method begins with operation 1252 where a fluid sample that is to be examined is provided. After operation 1252, the method advances to operation 1254 which separates component(s) of the fluid sampled by using one of isoelectric focusing, electrophoresis, etc. Then operation 1256 identifies the separated component(s) by infrared spectroscopy.

FIG. 19 illustrates a flowchart 1300 which defines a method where samples are analyzed using the multiple sample analyzing system in accordance with one embodiment of the present invention. In one embodiment, the method begins with operation 1302 which sets up the experiment. In one embodiment, variables such as, for example, time, voltage applied to the capillaries, temperature, etc. may be adjusted by inputting the setup variables into a graphical user interface in a computer that is attached to the multiple sample analyzing system.

After operation 1302, the method advances to operation 1304 which a sample holder and a source plate are placed in the multiple sample analyzing system. In one embodiment, the sample holder may be a wafer with a plurality of capillaries to hold the samples to be analyzed. In one embodiment, the sample holder may include an identification marking such as, for example, a bar code, RF ID, etc. In one embodiment, either or both of the wafer or the wafer holder may have marking(s) to identify the wafer. In addition, the source plate may also have an identification marking that may be inputted the computer. Therefore, the computer can recognize the source plate and the samples inside the particular wells of the source plate.

Then operation 1306 transfers the sample(s) from the source plate to the sample holder. In one embodiment, the write head can remove sample(s) from the source plate and inputs the sample(s) to the capillaries defined in the sample holder.

After operation 1306, the method moves to operation 1308 which applies a read head to the sample holder.

Then operation 1310 stabilizes temperature and environment inside the multiple sample analyzing system. Operation 1310 is an optional operation that may or may not be utilized. Stabilizing of the temperature and the environment may make the sample analysis process more controlled and consistent.

After operation 1310, operation 1312 executes setup data to run the experiment. In one embodiment, any suitable type of setup data may be executed. Examples of setup data execution can include, for example, control of the temperature and/or environment of the active region, reading of operating conditions and by the processor and adjusting of those conditions, application of voltage, the time when recording of data by the camera begins and/or ends, etc.

Then the method proceeds to operation 1314 which runs the experiment. After operation 1314, the method moves to operation 1316 which determines if there are any more experiments to run. If there are more experiments to run, the method returns to operation 1304 and repeats operations 1304, 1306, 1308, 1310, 1312, 1314, and 1316. In one embodiment, when operation 1316 determines that there are more experiments to run, another sample may be processed or a new sample may be loaded. If there are no more experiments to run the method ends.

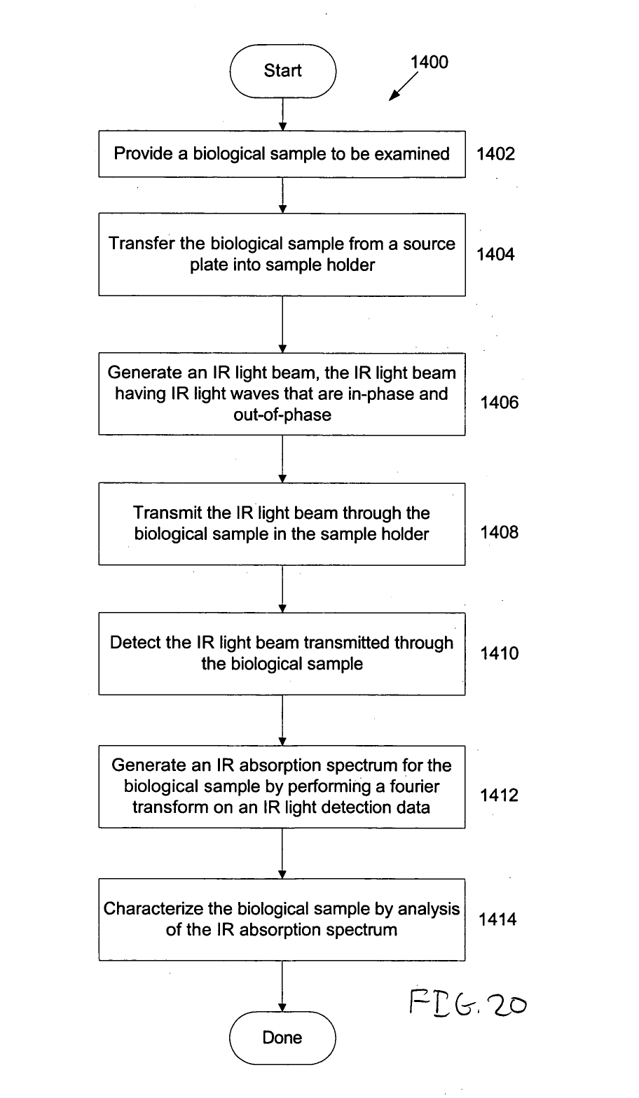

FIG. 20 depicts a flowchart 1400 which defines a detailed process whereby a biological sample is examined and identified in accordance with one embodiment of the present invention. In one embodiment, the flowchart 1400 begins with operation 1402 which provides a biological sample to be examined. After operation 1402, the method moves to operation 1404 which transfers the biological sample from a source plate into a sample holder. In one embodiment, the biological sample may be transferred into a recess defined on an active surface of a wafer. Then operation 1406 generates an IR light beam, the IR light beam having IR light waves that are in-phase and out-of-phase. After operation 1406, the method proceeds to operation 1408 which transmits the IR light beam through the biological sample in the sample holder. Then operation 1410 detects the IR light beam transmitted through the biological sample. After operation 1410, the method moves to operation 1412 which generates an IR absorption spectrum for the biological sample by performing a Fourier transform on an IR light detection data. Then operation 1414 characterizes the biological sample by analysis of the IR absorption spectrum.

While this invention has been described in terms of several preferred embodiments, it will be appreciated that those skilled in the art upon reading the preceding specifications and studying the drawings will realize various alterations, additions, permutations and equivalents thereof. It is therefore intended that the present invention includes all such alterations, additions, permutations, and equivalents as fall within the true spirit and scope of the claimed invention.

1. A silicon substrate for enabling the analysis of a biological sample, the semiconductor substrate comprising:
the silicon substrate having an active surface and a backside surface, the active surface of the silicon substrate having a plurality of recessed regions defined therein, the recessed region having a probe region on one side of the recessed region, and each one of the plurality of recessed regions being defined as an elongated well which is substantially rectangular, and the plurality of recessed regions configured for receiving a plurality of biological samples, and a complimentary probe region being on an opposite side of each one of the plurality of recessed regions, and a sample receiving region being between the probe region and the complimentary probe region, and the sample receiving region being capable of receiving the biological sample for analysis, and the complimentary probe region being capable of interfacing with an electrically conductive probe for enabling the analysis;
wherein the silicon substrate is disposed on a substrate holder and the silicon substrate is between about 1 micron and 4 cm thick and each one of the plurality of recessed regions being defined as a rectangular well having a length of about 5 mm, a width of about 125 microns, and a depth of about 25 microns, and the plurality of recessed regions including 10 capillaries.
